(19) United States
(12) Patent Application Publication
(10) Pub. No.: US 2014/0315754 A1
(43) Pub. Date: Oct. 23, 2014

(54) METHODS AND COMPOSITIONS FOR ASSESSING AND TREATING CANCER

(71) Applicants: Pomila Singh, Houston, TX (US); Shubhashish Sarkar, Galveston, TX (US); Malaney O'Connell, Galveston, TX (US)

(72) Inventors: Pomila Singh, Houston, TX (US); Shubhashish Sarkar, Galveston, TX (US); Malaney O'Connell, Galveston, TX (US)

(73) Assignee: The Board of Regents of the University of Texas System, Austin, TX (US)

(21) Appl. No.: 14/246,090

(22) Filed: Apr. 5, 2014

Related U.S. Application Data
(60) Provisional application No. 61/809,265, filed on Apr. 5, 2013.

(51) Int. Cl. C12Q 1/68; C12Q 1/66

(52) U.S. Cl. CPC C12Q 1/6897; C12Q 1/66
USPC 506/10; 435/320.1; 435/366; 435/6.13; 435/369

(57) ABSTRACT

Embodiments are directed to methods and compositions for determining the level of DCLK1-S in a sample and treating a subject having elevated levels of DCLK1-S.

### PRIORITY PARAGRAPH

This application claims the benefit of priority to U.S. Provisional Patent Application Ser. No. 61/809,265 filed Apr. 5, 2013, which is incorporated herein by reference in its entirety.

### STATEMENT REGARDING FEDERALLY FUNDED RESEARCH

This invention was made with government support under R01CA09795909 awarded by the National Institutes of Health. The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

Sporadic colorectal cancers (CRC) are the 3^{rd} most prevalent cancers in the US (Siegel et al. 2013. CA Cancer J Clin 2013, 63:11-30). Besides surgical removal, current standard of care remains chemotherapy/radiation therapy. It is becoming evident that cancer stem cells (CSCs) are resistant to these therapies and cause relapse of the disease (Ning et al. Cancer Biol Ther 2013, 14:295-303). Thus targeting CSCs, besides conventional therapies and/or available adjuvant therapies, is viewed as a treatment strategy for the future.

Normal colonic crypts are composed of cells which undergo continuous regeneration to replenish rapidly differentiating/dying cells (half-life: 4-6 days), as they are sloughed into the lumen (Milla J Pediatr Gastroenterol Nutr 2009, 48 Suppl 2:S43-5). Normal stem cells (NSCs), within a niche at the base of the crypt, self-renew and proliferate asymmetrically, giving rise to multi-potent/progenitor cells, which differentiate into functional lineages (Nicolas et al. PLoS Comput Biol 2007, 3:328; van der et al. Annu Rev Physiol 2009, 71:241-60). NSCs thus replenish and maintain a functioning colonic crypt, by balancing proliferation and differentiation; failure to differentiate results in hyperproliferation, which can lead to emergence of CSCs and tumor formation (Pinto et al. Bio Cell 2005, 97:185-86; Quante and Want Nat Rev Gastroenterol & Hepatol 2009, 6:724-37). A number of markers are used to identify intestinal NSCs, including Lgr5, CD44, DCLK1 (Barker et al. Nature 2007, 449:1003-7; May et al. Stem Cells 2008, 26:630-7; May et al. Stem Cells 2009, 27:2571-9; Sarkar et al. Gastroenterology 2011, 140:583-95, 595.e4). Lgr5 positive cells give rise to all the differentiated lineages (Barker et al. Nature 2007, 449:1003-7; Barker et al. Cold Spring Harb Symp Quant Biol 2008, 73:351-6). DCLK1 positive cells also form differentiated lineages in vivo (May et al. Stem Cells 2009, 27:2571-9; Sureban et al. Gastroenterology 2009, 137:649-59, 659.e1-2).

### SUMMARY

Colonic tumors increasingly express progastrins (PG) as they progress through hyperproliferative (Hp)/adenoma (Ad)/adenocarcinoma (AdCA) sequence. Autocrine PG up-regulates expression of stem-cell-markers DCLK1/CD44/LGR5. DCLK1 is doublecortin-like kinase 1. The DCLK1 gene encodes a protein that contains two N-terminal doublecortin domains, which bind microtubules and regulate microtubule polymerization, a C-terminal serine/threonine protein kinase domain, which shows substantial homology to Ca2+/calmodulin-dependent protein kinase, and a serine/proline-rich domain in between the doublecortin and the protein kinase domains, which mediates multiple protein-protein interactions. The microtubule-polymerizing activity is independent of its protein kinase activity. The encoded protein is involved in several different cellular processes, including neuronal migration, retrograde transport, neuronal apoptosis and neurogenesis. Multiple transcript variants are generated by alternative promoter usage and alternative splicing has been reported. These variants encode different isoforms, which are differentially expressed and have different kinase activities. Two major isoforms (short and Long) of DCLK1 (DCLK1-S, DCLK1-L) are expressed by neuroprogenitor cells and colonic epithelial cells.

The inventors have discovered that normal colon/non-tumorigenic cells (NCM460, HEKC) express DCLK1-L transcripts, transcribed from 5′ promoter, while tumorigenic (HEKmGAS) and colon-cancer cells/adenocarcinomas express DCLK1-S transcripts, transcribed from a promoter within intron V (FIG. 9B), indicating the mechanisms mediating up-regulation of S-isoform are specific to transformed/cancer cells.

Based on the discovery of differential expression of DCLK1 isoforms in normal stem cells compared to that in cancer stem cells, compositions and methods were developed for identifying compounds to specifically target either the expression of S isoform, or target the biological effects of cancer-specific S isoform, while sparing DCLK1-L functions in NSCs/normal tissues.

Certain embodiments are directed to recombinant nucleic acids related to DCLK1 isoforms and their downstream targets. In certain aspects reporter constructs specifically responsive to a biological pathway including a DCLK1 isoform are provided. In further aspects the isoform specific promoter regions are cloned into an expression cassette and positioned 5′ of a reporter gene. The expression of the reporter gene is controlled by the isoform specific promoter region, i.e., promoter is operatively coupled to the reporter gene. The expression cassettes can be temporarily or permanently incorporated into a cell or cell line, which can be used in a number of assays for detecting modulation of the isoform specific pathways.

In certain embodiments a recombinant nucleic acid comprises an expression cassette having a DCLK1-L promoter driving the expression of a heterologous gene. The heterologous gene can encode a reporter, such as a luciferase protein or a fluorescent protein. In certain aspects the expression cassette is comprised in a DNA vector. In a further aspect the DNA vector is a plasmid vector. In still further aspects the DNA vector encodes a selectable marker. In certain aspects the DCLK1-L promoter has a nucleic acid sequence of SEQ ID NO:1 that is operatively coupled to a reporter gene. In a further aspect the DCLK1-L promoter has a nucleic acid sequence of nucleotide 1202 to 4000 of SEQ ID NO:1, nucleotides 2277 to 4000 of SEQ ID NO:1, or nucleotides 3109 to 4000 of SEQ ID NO: 1. Certain embodiments include a cell comprising a DCLK1-L promoter expression cassette. In certain aspects the cell is a stably transfected cell.

In certain embodiments a recombinant nucleic acid comprises an expression cassette having a DCLK1-S intron V promoter driving the expression of a heterologous gene. The heterologous gene can encode a reporter, such as a luciferase protein or a fluorescent protein. In certain aspects the expression cassette is comprised in a DNA vector. In a further aspect the DNA vector is a plasmid vector. In still further aspects the DNA vector encodes a selectable marker. In certain aspects the DCLK1-S intron V promoter has the nucleic acid sequence of SEQ ID NO:2. In certain aspects the DCLK1-S intron V promoter has a nucleic acid sequence of nucleotides 1497 to 3604 of SEQ ID NO:2, nucleotides 1497 to 3229 of SEQ ID NO:2, nucleotides 2652 to 3604 of SEQ ID NO:2, or nucleotides 2652 to 3229 of SEQ ID NO:2. Certain embodiments include a cell comprising a DCLK1-S intron V promoter expression cassette. In certain aspects the cell is a stably transfected cell.

In certain embodiments a reporter construction will be based on genes regulated by, i.e., downstream of, DCLK1. These reporter constructs will include the DCLK1 responsive promoter segments of the DCLK1 target genes. These target genes may be directly or indirectly regulated by a DCLK1 protein. In certain aspects the genes are directly or indirectly regulated by DCLK1-S or DCLK1-L. In certain aspects a recombinant nucleic acid comprises an expression cassette having a COL3A1, ZEB2, NFATC2, TNFRS6B, or SPARC promoter region having a nucleic acid sequence of SEQ ID NOs:3, 4, 5, 6, or 7, respectively. In certain aspects these promoter regions are operatively coupled to a reporter gene. Certain embodiments are directed to a cell comprising these downstream reporter constructs.

Further embodiments are directed to a recombinant cell comprising an expression cassette that can express an inhibitory RNA under appropriate conditions. The expression cassette can comprise a regulatable promoter that can be turned on or off. In certain aspects the inhibitory RNA is an shRNA that is specific for an isoform of DCLK1. In a further aspect the inhibitory RNA is specific for DCLK1-S. In certain aspects the expression cassette is comprised in a lentiviral vector or other attenuated viral vectors. Infectious particles for expressing these vectors in mammalian cells can also be made.

Certain embodiments are directed to methods and composition for assessing DCLK1 activity. In certain aspects methods for assaying DCLK1-S activity are provided. In certain aspects a method for identifying the specific DCLK1-S inhibitor comprises (a) contacting a cell comprising a DCLK1-S reporter construct with a candidate compound; (b) assaying the cell for expression of the reporter construct; and (c) identifying a compound as an inhibitor of DCLK1-S if the expression of the reporter construction is significantly decreased as compared to control cell comprising a DCLK1-S reporter construct that is not exposed to the candidate compound.

In certain aspects, oligonucleotide primer pairs are designed to amplify short (S) and long (L) amplicons specific for a DCLK1 isoform. In a further aspect the primers can be used in conjunction with qRT-PCR. Colon cancer cells either express only S isoform (HCT-116) or both S and L forms of DCLK1 (HT-29, DLD1). In HEK293 cells, used as a model of PG responsive non-transformed cells, PG significantly up-regulated levels of both the transcripts.

The L-isoform is transcribed by the 5′ promoter of the gene while S-isoform is transcribed by an intron V promoter. Certain aspects are directed to promoter-reporter constructs. Promoter-reporter constructs specific to the two promoters were generated, and PG activation of both the promoter-constructs was confirmed in HEK293 cells.

Colonic tumors obtained from patients giving consent at the time of colonoscopy, were immunohistochemically analyzed, using an antibody that recognizes both forms of DCLK1. Hp/Ad/AdCA samples increasingly expressed DCLK1. Relative expression of S vs L transcripts was examined in stages I-III AdCAs, using a commercial cDNA plate. The S-isoform is increasingly up-regulated by 5-20-fold in stages I-III AdCAs, compared to that in surrounding normal tissues; the L-isoform, on the other hand, was only slightly elevated in tumor samples. Thus even though PG can potentially up-regulate expression of both S and L isoforms, the L-isoform was silenced in the tumors in a stage-dependent manner. This possibility was confirmed in established colon cancer cell lines. Thus, elevated levels of the S form is indicative of later stage cancers. Elevated levels of S form can be used as an indicator that more aggressive cancer therapy is warranted, or an inhibitor against the S isoform needs to be used.

Studies suggest that DCLK1 expression is required for maintaining proliferation of colon cancer cells and that while normal colonic cells/non-transformed HEK293 cells mainly express the L-isoform, colonic tumors increasingly express the S-isoform, suggesting a differential role of these isoforms in the normal vs cancer cell biology. Thus over-expression of DCLK1-S/PG may serve as robust diagnostic/prognostic markers, and provide useful targets for treating cancer-stem-cells, while sparing normal stem cells (which largely express the L-isoform and do not express PG).

Embodiments are directed to primers and combinations of primers for amplifying short DCLK1 and/or long DCLK1. In certain aspects primers that specifically amplify long DCLK1 include a forward primer comprising the sequence 5′-ggagtggtgaaacgcctgtac-3′ and a reverse primer comprising the sequence 5′-ttccattaactgagctgg-3′. In certain aspects primers that specifically amplify short DCLK1 include a forward primer comprising the sequence 5′-aacactaagactgtgtccatgt-3′ and a reverse primer comprising the sequence 5-′aagccttcctccgacacttct-3′. These primers can be used in amplification reactions to assess the expression levels of the short and/or long DCLK1 gene. In certain aspects the DCLK1 primers can be used to assess colon samples and to stage cancer cells present in the colon samples.

Certain embodiments are directed to identifying adenomacarcinoma by measuring and detecting a 5 to 20 fold or more increase in DCKL1-S relative to control.

Certain embodiments are directed to methods of amplifying S and/or L DCLK1 nucleic acids in a sample. The method comprising contacting a sample comprising nucleic acid of a cell expressing the DCLK1 gene with the S and/or L amplification primers under conditions that result in the amplification of segments of the S and/or L DCLK1 transcript or a nucleic acid defined by the S and/or L primers. The methods can further comprise analyzing or measuring the amplification products. In certain aspects the amplification is a quantitative or semi-quantitative polymerase chain reaction (qPCR).

Other embodiments are directed to methods of assessing the levels of the DCLK1 proteins, short and/or long forms, by contacting a sample containing a DCLK1 protein with a DCLK1 binding agent and measuring the levels of S or L DCLK1 in the sample. Those samples having an elevated level of the S form are determined to have a transformed phenotype indicating the need for more aggressive cancer therapy.

A “biological sample” in terms of the invention means a sample of biological tissue or fluid. Examples of biological samples are sections of tissues, blood, blood fractions, plasma, serum, urine or samples from other peripheral sources. A biological sample may be provided by removing a sample of cells from a subject, but can also be provided by using a previously isolated sample. For example, a tissue sample can be removed from a subject suspected of having a disease by conventional biopsy techniques. In one embodiment, the blood or tissue sample is obtained from the subject prior to initiation of radiotherapy, chemotherapy or other therapeutic treatment.

“Polynucleotide,” synonymously referred to as “nucleic acid molecule” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, double-stranded, or a mixture of single- and double-stranded regions. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

“Polypeptide” refers to any peptide or protein comprising amino acids joined by peptide bonds or modified peptide bonds.

“Antibody” refers to all isotypes of immunoglobulins (IgG, IgA, IgE, IgM, IgD, and IgY) including various monomeric and polymeric forms of each isotype, unless otherwise specified. Antibodies or functional fragments can be generated from any species. The antibodies or functional fragments thereof described herein can be labeled or otherwise conjugated to various chemical or biomolecule moieties, for example, for therapeutic or diagnostic or detection or treatment applications. The moieties can be cytotoxic, for example, bacterial toxins, viral toxins, radioisotopes, and the like. The moieties can be detectable labels, for example, fluorescent labels, radiolabels, biotin, and the like, which are known in the art.

The terms “treating” or “treatment” refer to any success or indicia of success in the attenuation or amelioration of a pathology or condition, including any objective or subjective parameter such as abatement, remission, diminishing of symptoms or making the pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

As used herein, the term “subject” refers to any mammal, including both human and other mammals. In certain aspects the methods of the present invention are applied to human subjects.

Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

“Prognosis” refers to a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable responses to cancer treatments, such as a conventional cancer therapy. A good or bad prognosis may, for example, be assessed in terms of patient survival, likelihood of disease recurrence, disease metastasis, or disease progression (patient survival, disease recurrence and metastasis may for example be assessed in relation to a defined time point, e.g. at a given number of years after cancer surgery (e.g. surgery to remove one or more tumors) or after initial diagnosis). In one embodiment, a good or had prognosis may be assessed in terms of overall survival, disease-free survival or progression-free survival.

In one embodiment, the marker (DCKL1-S or L) level is compared to a reference level representing the same marker. In certain aspects, the reference level may be a reference level of expression from non-cancerous tissue from the same subject. Alternatively, reference level may be a reference level of expression from a different subject or group of subjects. For example, the reference level of expression may be an expression level obtained from tissue of a subject or group of subjects without cancer, or an expression level obtained from non-cancerous tissue of a subject or group of subjects with cancer. The reference level may be a single value or may be a range of values. The reference level of expression can be determined using any method known to those of ordinary skill in the art. In some embodiments, the reference level is an average level of expression determined from a cohort of subjects with cancer. The reference level may also be depicted graphically as an area on a graph.

The reference level may comprise data obtained at the same time (e.g., in the same hybridization experiment) as the patient's individual data, or may be a stored value or set of values e.g. stored on a computer, or on computer-readable media. If the latter is used, new patient data for the selected marker(s), obtained from initial or follow-up samples, can be compared to the stored data for the same marker(s) without the need for additional control experiments.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates the location and design of S and L primers in DCLK1-L (SEQ ID NO NO:8) and DCLK1-S(SEQ ID NO:9).

FIG. 2 illustrates representative steps used to conduct nucleic acid amplification methods.

FIG. 3 illustrates representative steps for conducting S and L DCLK1 immunohistochemistry on tumor tissues.

FIG. 4 illustrates expression of DCLK1-L and DCLK1-S nucleic acid in human colon cancer cells.

FIG. 5 illustrates an effect of progastrin expression, progastrin increases both DCLK1-L and DCLK1-S in HEK-mGAS Cells. But, Long iso-form is silenced in majority of colon cancers, and thus PG expression is unable to up-regulate the expression of long isoform in situ in tumors silenced at the CpG site in the 5′ promoter of the long isoform (Analyzed by qRT-PCR).

FIG. 6 illustrates an immunohistochemical analysis of expression of progastrin (C, D, G, H) and DCLK1 (A, B, E, F) by Normal (A, B, C, D) vs Colonic adenomatous Tumors (benign polyps) (E, F, G, H) from patients, obtained at the time of endoscopy via our approved IRB protocol. FIG. 6I illustrates the quantitation of area stained.

FIG. 7 diagram of the protein structure of short (SEQ ID NO:16) and long (SEQ ID NO:15) isoform.

FIG. 8 illustrates that expression of both DCLK1-L and S was significantly increased in colonic adenocarcinomas from patients. (Analyzed by qRT-PCR).

FIG. 9 (A) Western Blot (WB) of HEKC (non-transformed) and HEKmGAS (transformed) cells with DCLK1-antibody. (B) (i) DCLK1 gene transcribing L and S isoforms; (ii) homology between transcripts of L/S isoforms of DCLK1 (shaded) Red lines at 5′ and represent non-homologous sequences; (iii) protein structure of L and S isoforms of DCLK1. (C) DCLK1 expression (S/S) in normal and CRC samples from patients. NCM-460/HEKmGAS used as controls for L/S DCLK1, respectively.

FIG. 10 Effect of control-siRNA or DCLK1-siRNA on growth of HEK-C & HEKmGAS cells.

FIG. 11 (A) IF/IHC staining for DCLK1 in human samples from normal colons (NC) or tubular adenonmas (TA). (B) Amplification of S/L DCLK1 transcripts in cell lines by RT-PCR. (C) qRT-PCR of cDNA from CRC samples with DCLK1-S primers.

FIG. 12 (A) Methylation of CpG sites in 5′-promoter of DCLK1-gene in cell lines/human samples; NC=normal colon; CRC=colorectal cancer. (B) Expression of L/S DCLK1 in HCT-116 cells before (−) or after (+) treatment with 5-Aza-Cytidine by RT-PCR.

FIG. 13 (A) DCLK1 expression in ShC/ShD clones of HCT-116 cells (where in ShC clones express control vector and ShD clones are expressing effective shRNA against DCLK1) by WB analysis; (B) Growth of ShC/ShD clones in response to increasing serum concentration; (C) Spheroid Assay of ShC/ShD clones.

FIG. 14 HTS Bioassay: (A) RT-PCR analysis of COL3A1 in ShC and ShD clones (5′upstream nucleotide sequence of COL3A1 gene is provided in SEQ ID NO:10). (B) The map of the promoter constructs fused to secretable Cypridina Luciferase (C-Luciferase). (C) The flow diagram of the method used to measure the promoter activity of COL3A1 constructs in HCT116 cells. (D) The promoter activity of COL3A1 constructs in HCT116 cells. The loss of promoter activity in presence of shRNA to DCLK1 gene indicates the transcriptional control of COL3A1 promoter by DCLK1-S in HCT116 cells.

FIG. 15 Genes down stream of DCLK1-S in cancer cells, but not down stream of DCLK1-L in normal cells, as determined by using RNAseq data from the control and DCLK1-ShRNA expressing clones, and by applying ingenuity pathway analysis.

FIG. 16 (A) Promoter-reporter constructs designed for the 5′ promoter for use in bioassays for discovering inhibitors that target expression of S isoform but not L isoform. (B) Promoter-reporter constructs designed for the intron V promoter to use in bioassays for discovering inhibitors that target expression of S isoform but not L isoform.

### DESCRIPTION

Two major isoforms of DCLK1 (DCLK1-S, DCLK1-L) have been reported. Knock-down (KO) of DCLK1 in embryonic stem cells was lethal at pre-natal stages, suggesting a critical role of L-isoform during development (Dijkmans et al. Cent Ner Syst Agents Med Chem 2010, 10:32-46). It is now know that 5′ promoter of DCLK1 is epigenetically silenced in cholangiocarcinomas and CRCs, due to DNA methylation (Andresen et al. Epigenetics 2012, 7(11):1249-57; Vedeld et al. Epigenetics 2014, 2:9(3)), which explains an absence of DCLK1-L in CRC cells/adenocarcinomas. However, significant increase in DCLK1 expression has been reported in CRCs and pancreatic lesions, using commercially available antibodies against the common C-terminal end of DCLK1-L and DCLK1-S.

Hyperproliferation of colonic crypts in response to growth factors (such as progastrin, PG), up-regulates DCLK1 expression (Sarkar et al. Gastroenterology 2011, 140:583-95, 595.e4), and enhances colon carcinogenesis (Jin et al. J Clin Invest. 2009, 119(9):2691-701). Overexpression of PG in HEK-293 cells imparts tumorigenic/metastatic potential to HEKmGAS cells, which express DCLK1-S, unlike isogenic HEKC cells. DCLK1 is expressed by NSCs in intestinal crypts, albeit at much lower concentrations by a small population of quiescent stem cells.

DCLK1-L isoform transcription is driven by promoter upstream or in the 5′ segment of the DCLK1 gene, while DCLK1-S isoform transcription is driven by a promoter located in intron V of the DCLK1 gene (“intron V promoter”). Promoter-reporter constructs, specific to the two promoters, were generated to confirm promoter activation in response to PG in HEK293 cells. Colonic tumors obtained from consented patients at the time of colonoscopy, were immunohistochemically analyzed, using an antibody that recognizes both the forms of DCLK1; Hp/Ad/AdCA increasingly expressed DCLK1. Relative expression of the DCLK1-S compared to DCLK1-L transcripts was examined in stages I-III AdCAs, using a commercial cDNA plate.

As described herein, the DCLK1-S isoform is increasingly up-regulated by 5-20-fold in stages I-III AdCAs, compared to that in surrounding normal tissues. The DCLK1-L isoform, on the other hand, was only slightly elevated in most tumor samples. Thus even though PG can potentially up-regulate expression of both S and L isoforms, the L-isoform appears to be silenced in many tumors in a stage-dependent manner. Later stage cancers can be identified by elevated levels of the S form. In certain aspects lower expression of the L-isoform can be correlated to an increased methylation level of the 5′ or L-isoform promoter.

As described herein DCLK1 expression is required for maintaining proliferation of colon cancer cells and that while normal colonic cells/non-transformed HEK293 cells mainly express the L-isoform, colonic tumors increasingly express the S-isoform, suggesting a differential role of these isoforms in the normal versus cancer cell biology. Thus, over-expression of DCLK1-S/PG serves as a diagnostic/prognostic marker, and provides a useful target for identifying modulators of the DCLK1 pathway and treating cancer-stem-cells, while sparing normal stem cells (which largely express the L-isoform and do not express PG).

DCLK1-S, supports tumorigenic potential of cancer stem cells (CSCs) by up-regulating a unique set of genes, while the DCLK1-L expressed by normal stem cells (NSCs), regulates a separate set of genes for maintaining multipotency and asymmetrical division of NSCs. Thus, assays to develop inhibitors, which are developed to target the expression of down-stream genes will inhibit the biological effects of DCLK1-S on CSCs, and eliminate the growth of CSCs. Down-regulation of the expression of DCLK1-S by inhibiting the transcriptional activity of DCLK1 intron V promoter, but not the transcriptional activity of DCLK1 5′ promoter inhibits the growth of cancer stem cells but not the growth of normal stem cells.

### I. NUCLEIC ACID COMPOSITIONS

Certain embodiments described herein are directed to nucleic acids. In certain aspects the nucleic acids described comprise expression cassettes. In further aspects the expression cassettes comprise DCLK1 isoform specific promoters or promoters derived from genes up-regulated by DCLK1 pathways. The promoter can be configured in such a way as to be used in expressing detectable gene products that can be used to evaluate the activity of a promoter under particular conditions.

In certain aspects a 5′ DCLK1 promoter is used. The 5′ DCLK1 promoter can comprise the 4 kb of nucleic acid sequence upstream of the DCLK1-L start site (SEQ ID NO:1). In certain aspects the promoter is position to drive the expression of a detectable gene product or marker. In a further aspect the marker can be a pLightSwitch_Prom (3656 bp) nucleic acid utilizing RenSP (optimized luciferase gene).

In certain aspects an intron V DCLK1 promoter is used. The intron V DCLK1 promoter can comprise the 4 kb of nucleic acid sequence from intron V that is upstream of the start site for the DCLK1-S transcript (SEQ ID NO:2). In a further aspect the marker can be a pLightSwitch_Prom (3656 bp) nucleic acid utilizing RenSP (optimized luciferase gene).

The term “nucleic acid vector” is used to refer to a carrier nucleic acid molecule into which a nucleic acid sequence can be inserted for introduction into a cell where it can be replicated, transcribed, and/or translated (i.e., expressed). A nucleic acid sequence can be “exogenous,” which means that it is foreign to the cell into which the vector is being introduced or that the sequence is “endogenous” to the cell but in a position within the host cell in which the sequence is ordinarily not found. In certain aspects an exogenous vector can encode an endogenous nucleic acid, e.g., all or part of a endogenous gene. Nucleic acid vectors include plasmids, cosmids, viral genomes, and other expression vectors (bacteriophage, animal viruses, and plant viruses), artificial chromosomes (e.g., YACs), and the like.

The term “expression vector” refers to any type of genetic construct comprising a nucleic acid coding for an RNA capable of being transcribed. In some cases, RNA molecules are then translated into a protein, polypeptide, or peptide. In other cases, these sequences are not translated, for example, in the production of inhibitory RNA, antisense molecules, or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described herein.

Certain aspects involve the use of promoter regions of one or more genes. Examples of promoters include those nucleic acid sequences of SEQ ID NOs 1, 2, 3, 4, 5, 6, or 7. In certain aspects the nucleic acid comprise a nucleotide sequence that is 95, 98, or 100% identical to SEQ ID NO:1, 2, 3, 4, 5, 6, or 7, yet still provides certain identifiable transcriptional control characteristics of the endogenous gene from which the promoter is naturally associated.

A. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” operatively coupled”, “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the RNA. The spacing between promoter elements frequently is flexible, so that promoter function is preserved when elements are inverted or moved relative to one another. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous” or “homologous.” Similarly, an enhancer may be one naturally associated with a nucleic acid sequence, located either downstream or upstream of that sequence. Alternatively, certain advantages will be gained by positioning the nucleic acid under the control of a recombinant, exogenous, or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from a virus, or prokaryotic or eukaryotic cell, and promoters or enhancers not “naturally occurring,” i.e., containing different elements of different transcriptional regulatory regions, and/or mutations that alter expression.

Naturally, it will be important to employ a promoter and/or enhancer that effectively directs the expression of the DNA segment in the organelle, cell, tissue, organ, or organism chosen for expression. The promoters employed may be constitutive, tissue-specific, inducible, and/or useful under the appropriate conditions to direct expression of a DNA segment. The promoter may be heterologous or endogenous.

Additionally any promoter/enhancer combination (as per, for example, the Eukaryotic Promoter Data Base EPDB, world-wide-web at epd.isb-sib.ch/) could also be used to drive expression. Use of a T3, T7, or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

In certain aspects, a nucleic acid of the invention can comprise a non-inducible or inducible promoter that will be expressed specifically in CSCs. Such non-inducible promoters include cell-specific DCLK1 promoters or promoter from DCLK1-S or DCLK1-L regulated genes. Such inducible promoters include promoters under the control of a response element that is inducible by chemical, peptide, ligand, or metabolites.

B. Initiation Signals

A specific initiation signal also may be used or required for efficient translation of coding sequences. These signals include an ATG initiation codon or adjacent sequences. Exogenous translational control signals may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

C. Post-Transcriptional Regulatory Elements (PRE)

Post-transcriptional regulation is the control of gene expression at the RNA level, i.e., between the transcription and the translation of the gene. In certain aspects, the Woodchuck Hepatitis Virus Post-transcriptional Regulatory Element (WPRE) is used. WPRE increases the levels of nuclear transcripts and facilitates RNA export. WPRE may facilitate other steps in RNA processing, directing RNAs that would normally be degraded within the nucleus to be efficiently expressed. The WPRE can also function to facilitate the generation of RNA-protein complexes that would protect newly synthesized transcripts from degradation in the nucleus. (Zufferey et al., Journal of Virology, 73: 2886-2892, 1999 and U.S. Pat. No. 6,284,469, which is incorporated herein by reference).

D. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

E. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

F. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers include reporter genes or screenable markers. A “reporter gene” or “reporter sequence” refers to any sequence that produces a protein product that is easily measured, preferably although not necessarily in an assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. Screenable enzymes such as herpes simplex virus thymidine kinase (tk) or chloramphenicol acetyltransferase (CAT) may be utilized. One of skill in the art would also know how to employ immunologic markers, possibly in conjunction with fluorescence assisted cell sorting (FACS) and/or immunohistochemistry. The marker used is not believed to be important, so long as it is capable of being expressed under the appropriate conditions. Further examples of selectable and screenable markers are well known to one of skill in the art.

In certain embodiments an expression vector can be used to provide an inhibitory RNA to a cell in vitro or in vivo. DCLK1-siRNA, which targets both isoforms, causes significant loss in proliferation of both tumorigenic (HEKmGAS) and non-tumorigenic (HEKC) cells (FIG. 10), suggesting L isoform maintains NSCs while S isoform maintains CSCs. The down-regulation of DCLK1 in CRC cells, which only express DCLK1-S, causes loss of 2D and 3D (spheroidal) growths (Kantara et al. Cancer Res. 2014 Mar. 13); DCLK1-siRNA, which targets both L and S iosforms has also been reported by the inventors and others to attenuate growth of CRC tumors in vivo (Sureban et al. Gastroenterology 2009, 137:649-59, 659.e1-2; Kantara et al. Cancer Res. 2014 Mar. 13; Sureban et al. Journal of Nanobiotechinology 2011, 9:40). The inventors discovered that DCLK1 positive CSCs are resistant to chemopreventative agents, unlike Lgr5 positive stem cells; DCLK1 positive cancer cells, treated with curcumin underwent autophagic survival and re-grew (relapse) as spheroids, in an in vitro relapse assay (Kantara et al. Cancer Res. 2014 Mar. 13). Treatment with DCLK1-siRNA/shRNA eliminated relapse/re-growth of spheroids from CSCs (Kantara et al. Cancer Res. 2014 Mar. 13), while treatment with Lgr5/CD44 siRNAs was not as effective. Thus accumulating evidence from literature and from our laboratory strongly supports a critical role of DCLK1 in cancer stem cell biology. In certain aspects, components are provided to a CSC or organ or tissue by using nucleic acids that encode or express an RNA that specifically inhibits DCLK1-S. Viral and non-viral delivery vectors can be used to delivery inhibitory RNA to a cell or organ.

The ability of certain viruses to infect cells or enter cells via receptor-mediated endocytosis, and to express virally encoded genes have made them attractive candidates for the transfer of foreign nucleic acids into cells (e.g., mammalian cells). Viruses may thus be utilized that encode and express agents to decrease the activity of DCLK1-S transcription or activity, and increase transcription of genes that are down-regulated by DCLK1-S.

Retroviral Vectors.

Retroviruses have the ability to integrate their genes into the host genome, transferring a large amount of foreign genetic material, infecting a broad spectrum of species and cell types and of being packaged in special cell-lines. In order to construct a retroviral vector, a nucleic acid (e.g., one encoding a protein of interest) is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136, each of which is incorporated herein by reference). Some examples of lentivirus include the Human Immunodeficiency Viruses: HIV-1, HIV-2 and the Simian Immunodeficiency Virus: SIV. Lentiviral vectors have been generated by attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, which is incorporated herein by reference.

Certain embodiments are directed to the constructs and cell lines described in the following table.

| Cell Lines/Construct | | Name | Description |
|---|---|---|---|
| Luciferase Promoter | Plasmids | 5′ promoter | Promoter Constructs shown in FIG. |
| Reporter Constructs | | for DCLK1-L | 16A for DCLK1-L in pGL2-Basic |
| | | | Vector (Promega) |
| | | DCLK1-S | Promoter Constructs shown in FIG. |
| | | Intron-V | 16B in pGL2-Basic Vector |
| | | Promoter | (Promega) for DCLK1-S |
| | | | AL160392.12 |
| Luciferase Promoter | Plasmids | COL3A1 | Promoter construct in pCLuc- |
| Reporter Constructs | (Bioassay) | | Basic2 Vector (New England |
| | | | Biolab). Primers located at −1394/ |
| | | | +68R and −1184F/+68R |
| | | | located in NC_000002.12 |
| Stable expression of | Cell lines | DCLKL- | Promoter construct in pCLuc- |
| promoter-Luciferase reporter | | Clones | Basic2 Vector (New England |
| constructs in human cancer | | | Biolab). Primers located at −1783/−530 |
| cell lines and in human | | | in AL160392.12. CLONE |
| immortalized normal cell | | | DCLKL1L |
| lines | | | |
| Stable expression of | Cell lines | DCLK1S- | Promoter construct in pCLuc- |
| promoter-Luciferase reporter | | Clones | Basic2 Vector (New England |
| constructs in human cancer | | DCLK1S- | Biolab). Cloned from |
| cell lines and in human | | Clone | AL160392.12. CLONE |
| immortalized normal cell | | | Name: DCLK1S |
| lines | | | Promoter construct in pCLuc- |
| | | | Basic2 Vector (New England |
| | | | Biolab). Cloned from |
| | | | AL160392.12. CLONE |
| | | | Name: DCLK1S |
| Stable expression of | Cell lines | COL3A1- | Promoter construct in pCLuc- |
| promoter for COL3A1- | | Clone | Basic2 Vector (New England |
| Luciferase reporter | | COL3A1- | Biolab). Cloned from |
| constructs in human cancer | | Clone | NC_000002.12. CLONE |
| cell lines and in human | | | Name: COL3A1 |
| immortalized normal cell | | | Promoter construct in pCLuc- |
| lines | | | Basic2 Vector (New England |
| | | | Biolab). Cloned from |
| | | | NC_000002.12. CLONE |
| | | | NameOL3A1 |
| Stable expression of ZEB2 | Cell lines | ZEB2-Clone | Promoter construct in pCLuc- |
| promoter-Luciferase reporter | | ZEB-Clone | Basic2 Vector (New England |
| constructs in human cancer | | | Biolab). Cloned from |
| cell lines and in human | | | GRCh37:2:145277206:145279958 |
| immortalized normal cell | | | CLONEiName: ZEB2 |
| lines | | | Promoter construct in pCLuc- |
| | | | Basic2 Vector (New England |
| | | | Biolab). Cloned from |
| | | | GRCh37:2:145277206:145279958 |
| | | | CLONE Name: 1160ZEB2 |
| Stable expression of | Cell lines | NFATC2- | Promoter construct in pCLuc- |
| NFATC2 promoter- | | Clone | Basic2 Vector (New England |
| Luciferase reporter | | | Biolab). Cloned from |
| constructs in human cancer | | | GRCh37:20:50158609:50162258:- |
| cell lines and in human | | | CLONE Name: 2330NFATC2 |
| immortalized normal cell | | | CLONE Name: 867NFATC2 |
| lines | | | |
| Stable expression of | Cells lines | TNFRS6B- | Promoter construct in pCLuc- |
| TNFRS6B reporter- | | Clone | Basic2 Vector (New England |
| Luciferase reporter | | | Biolab). Primers located at −766/ |
| constructs in human cancer | | | +224R CLONE Name: |
| cell lines and in human | | | TNFRS6B |
| immortalized normal cell | | | |
| lines | | | |
| Stable expression of SPARC | Cell Lines | SPARC- | Promoter construct in pCLuc- |
| promoter-Luciferase reporter | | Clone | Basic2 Vector (New England |
| constructs in human cancer | | SPARC- | Biolab). Cloned from |
| cell lines and in human | | Clone | NC_000005.9 CLONE |
| immortalized normal cell | | | Name: SPARC |
| lines | | | Promoter construct in pCLuc- |
| | | | Basic2 Vector (New England |
| | | | Biolab). Cloned from |
| | | | NC_000005.9 CLONE |
| | | | Name: SPARC |
| Cancer Cell line clones as | Cell Lines | hSC | stably expressing empty vector |
| controls and for Stable down | | hSD1 | pGZIP Lentiviral vector |
| regulation of DCLK1-S | | hSD2 | stably expressing shrna sequence |
| expression | | hSD3 | ATCGTTCTGTTATTGTAGC in |
| | | | pGZIP Lentiviral vector |
| | | | stably expressing shrna sequence |
| | | | ATCGTTCTGTTATTGTAGC in |
| | | | pGZIP Lentiviral vector |
| | | | stably expressing shrna sequence |
| | | | ATCGTTCTGTTATTGTAGC in |
| | | | pGZIP Lentiviral vector |
| Bioassay for screening | | Bioassay with | Test clones for using in HTS |
| Inhibitors of DCLK1-S | | Test Clones of | screening |
| and/or its functions | | cancer cells as | |
| | | described in | |
| | | the text for the | |
| | | various | |
| | | promoter- | |
| | | reporter | |
| | | constructs | |
| | | shown above | |

### II. DIAGNOSTIC/PROGNOSTIC METHODS

Methods for detecting and/or measuring DCLK1 isoforms can be used as a biomarker for identifying patients at the risk for cancer based on biochemical assays and gene expression levels.

A marker or biomarker is an organic biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. As such, they are useful as markers for disease (diagnostics), therapeutic effectiveness of a drug (theranostics) and of drug toxicity.

In certain aspects, the markers (e.g., DCLK1-S and/or DCLK-L protein) of this invention can be measured or detected by immunoassay. Immunoassay requires biospecific capture reagents, such as antibodies, to capture the markers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies.

This invention contemplates traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

In particular embodiments of the invention, the expression of DCKL1 and/or DCLK1-S and/or DCLK-L in a sample is examined using immunohistochemistry and staining protocols. Immunohistochemical staining of tissue sections has been shown to be a reliable method of assessing or detecting presence of proteins in a sample. Immunohistochemistry (“IHC”) techniques utilize an antibody to probe and visualize cellular antigens in situ, generally by chromogenic or fluorescent methods.

For sample preparation, a tissue or cell sample from a mammal (typically a human patient) may be used. Examples of samples include, but are not limited to, cancer cells such as colon, breast, prostate, ovary, lung, stomach, pancreas, lymphoma, and leukemia cancer cells. The sample can be obtained by a variety of procedures known in the art including, but not limited to surgical excision, aspiration or biopsy. The tissue may be fresh or frozen. In one embodiment, the sample is fixed and embedded in paraffin or the like.

The tissue sample may be fixed (i.e. preserved) by conventional methodology (See e.g., “Manual of Histological Staining Method of the Armed Forces Institute of Pathology,” 3rd edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the sample is to be histologically stained or otherwise analyzed. One of skill in the art will also appreciate that the length of fixation depends upon the size of the tissue sample and the fixative used. By way of example, neutral buffered formalin, Bouin's or paraformaldehyde, may be used to fix a sample.

Generally, the sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., “Manual of Histological Staining Method of the Armed Forces Institute of Pathology”, supra). Examples of paraffin that may be used include, but are not limited to, Paraplast, Broloid, and Tissuemay. Once the tissue sample is embedded, the sample may be sectioned by a microtome or the like (See e.g., “Manual of Histological Staining Method of the Armed Forces Institute of Pathology”, supra). By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., “Manual of Histological Staining Method of the Armed Forces Institute of Pathology”, supra). Alternatively, commercially available deparaffinizing non-organic agents such as Hemo-De7 (CMS, Houston, Tex.) may be used.

Optionally, subsequent to the sample preparation, a tissue section may be analyzed using IHC. IHC may be performed in combination with additional techniques such as morphological staining and/or fluorescence in-situ hybridization. Two general methods of IHC are available; direct and indirect assays. According to the first assay, binding of antibody to the target antigen (e.g., DCLK1-S and/or DCLK-L) is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for immunohistochemistry typically will be labeled with a detectable moiety. Numerous labels are available which can be generally grouped into the following categories: (a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting. (b) Colloidal gold particles. (c) Fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above. The fluorescent labels can be conjugated to the antibody using the techniques disclosed in Current Protocols in Immunology, supra, for example. Fluorescence can be quantified using a fluorimeter. (d) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light, which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed. J. Langone & H. Van Vunakis), Academic press, New York, 73:147-166 (1981).

Examples of enzyme-substrate combinations include, for example: (i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); (ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase).

Numerous other enzyme-substrate combinations are available to those skilled in the art. For a general review of these, see U.S. Pat. Nos. 4,275,149 and 4,318,980. Sometimes, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten and one of the different types of labels mentioned above is conjugated with an anti-hapten antibody. Thus, indirect conjugation of the label with the antibody can be achieved.

Aside from the sample preparation procedures discussed above, further treatment of the tissue section prior to, during or following IHC may be desired, For example, epitope retrieval methods, such as heating the tissue sample in citrate buffer may be carried out (see, e.g., Leong et al. Appl. Immunohistochem. 4 (3):201 (1996)).

Following an optional blocking step, the tissue section is exposed to primary antibody for a sufficient period of time and under suitable conditions such that the primary antibody binds to the target protein antigen in the tissue sample. Appropriate conditions for achieving this can be determined by routine experimentation. The extent of binding of antibody to the sample is determined by using any one of the detectable labels discussed above. Preferably, the label is an enzymatic label (e.g. HRPO) which catalyzes a chemical alteration of the chromogenic substrate such as 3,3′-diaminobenzidine chromogen. Preferably the enzymatic label is conjugated to antibody that binds specifically to the primary antibody (e.g. the primary antibody is rabbit polyclonal antibody and secondary antibody is goat anti-rabbit antibody).

Specimens thus prepared may be mounted and cover slipped. Slide evaluation is then determined, e.g. using a microscope, and staining intensity criteria, routinely used in the art, may be employed. Where the antigen is DCLK1-S and/or DCLK-L protein, staining intensity criteria may be evaluated as follows:

Staining Pattern (Score), No staining is observed in cells (0), Faint/barely perceptible staining is detected in more than 10% of the cells (1+), Weak to moderate staining is observed in more than 10% of the cells (2+), Moderate to strong staining is observed in more than 10% of the cells (3+).

### III. DCLK1 RELATED BIOASSAYS

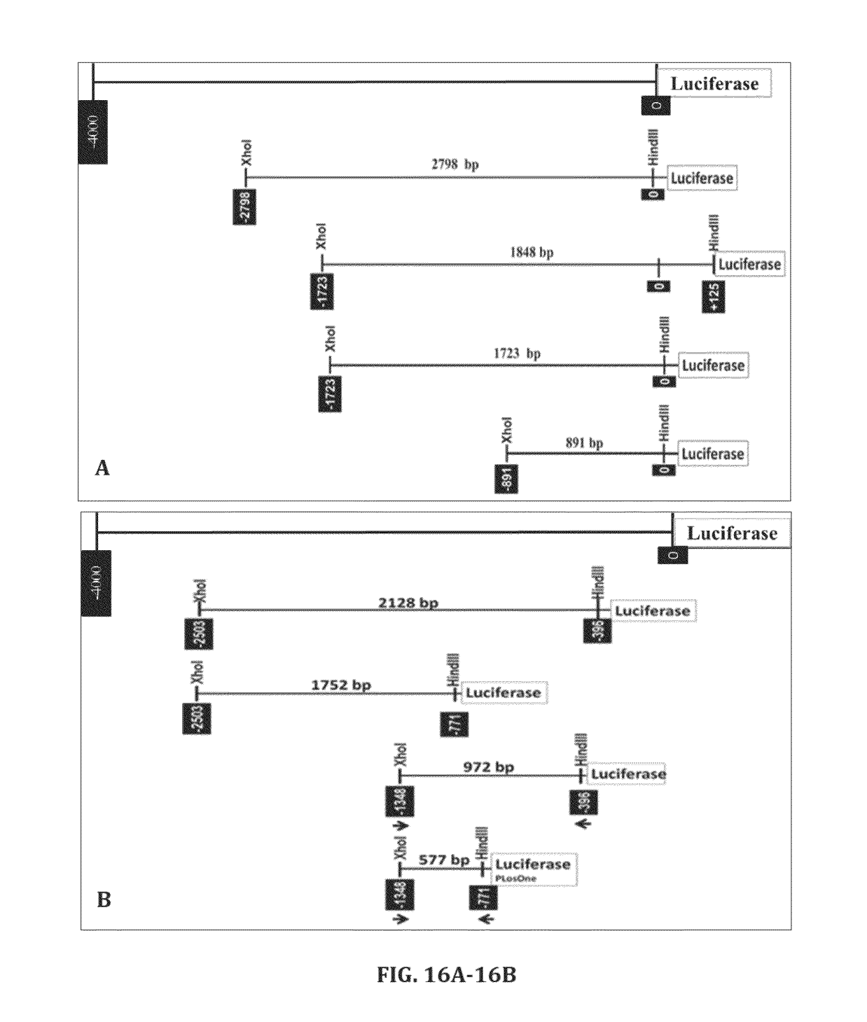

Bioassays with promoter-reporter constructs for the two isoforms of the DCLK1 gene. The 5′ and intron V promoter-reporter constructs have been made and cloned into host cells (e.g., cancer cells) for purposes of conducting high throughput screening (HTS) bioassays (for example see FIGS. 16A and 16B). These tools will allow the screening and identification of specific inhibitors that reduce the activation of intron V promoter (and thus the expression of DCLK1-S) without any effects on DCLK1-L.

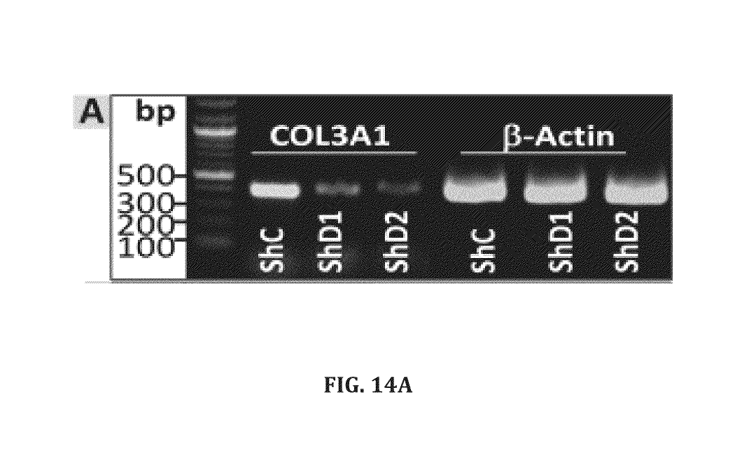

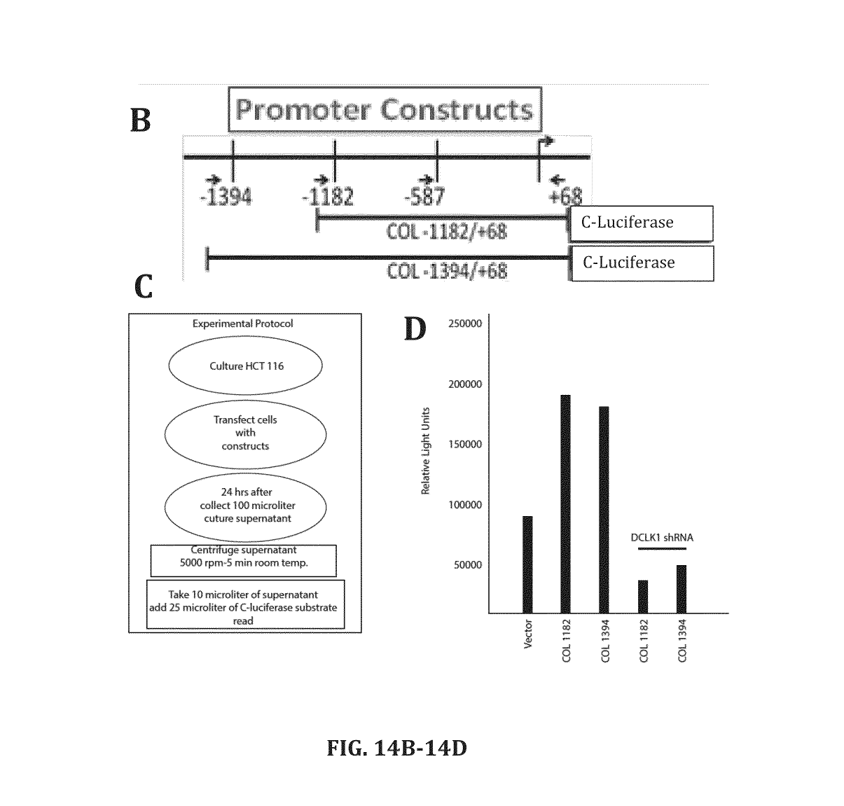

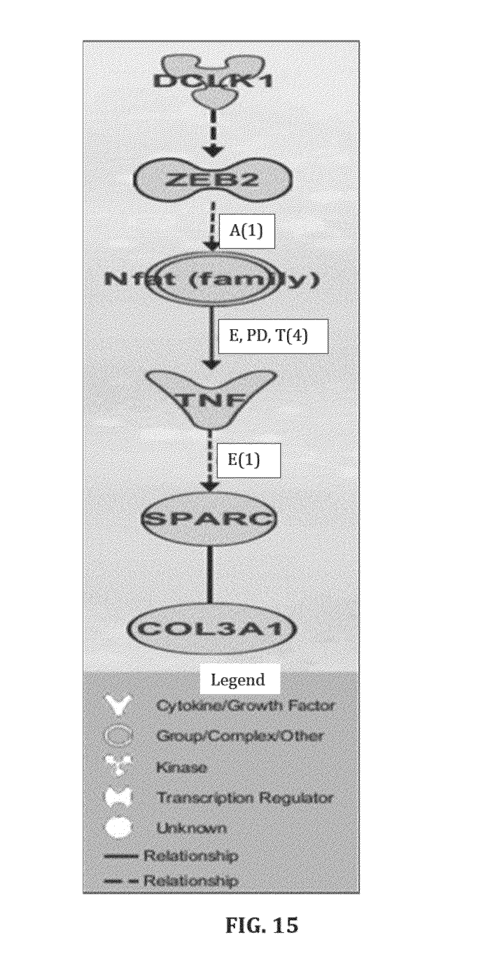

Promoter fragments of ˜1-4Kd, of identified genes, downstream of DCLK1-S(FIG. 15) were PCR amplified from gDNA of HCT-116 cells and cloned into pCLuc-BASIC-2 Vector. An advantage of this plasmid is that cypridina luciferase is a secreted protein, and thus the supernatant of the transfected cells is used for measuring luciferase, without lysing the cells, as shown in FIG. 14D, for a representative gene. The minimum promoter, required to measure maximum transcriptional activation of the gene in ShC clones of CRC cells, was used for generating stably-expressing clones of HCT-116 cells (named Test Clones for the specific gene). DCLK1-siRNA treatment of Test Clones results in loss of luciferase activity by >5-7fold, compared to control vector levels, confirming the validity of the developed bioassays (an example is shown for one of the genes in FIG. 14E).

Loss of luciferase activity in conditioned medium of Test Clones is being used as a readout of inhibitory efficacy against functions of S isoform. Initial filtering of the compounds is done in HCT-116 cells stably expressing cypridina luciferase, down-stream of CMV promoter. This filtering step will eliminate non-specific inhibition due to non-specific inhibition of transcriptional activation of reporter constructs or luciferase enzyme. These experiments will help us to discover lead compounds for developing inhibitory drugs for specifically targeting CSCs, while sparing NSCs, for all DCLK1-S dependent cancers.

### IV. KITS

In another aspect, the present invention provides kits for assaying DCLK1-S specific modulators or for assessing cancer status based on detecting DCLK1 activity or the expression of genes downstream from DCLK1-S. In one embodiment, the kit comprises a solid support, such as a chip, a microtiter plate or a bead or resin having a capture reagent attached thereon, wherein the capture reagent binds a biomarker of the invention. Thus, for example, the kits of the present invention can comprise mass spectrometry probes for SELDI, such as ProteinChip® arrays. In the case of biospecfic capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

Certain embodiments are directed to cell based reagents including reporter constructs and their host cells.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

### V. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### L and S Isoforms of DCLK1 Gene are Differentially Regulated in Normal Versus Cancer Stem Cells

Progastrin (PG) expression increases in Colonic tumors as growth progresses through Hyperproliferative(Hp)/adenoma(Ad)/adenomacarcinoma(AdCA) stages (Singh et al. Gastroenterology. 2005: 128(4). Supplement2; Do et al. Cancer Prev Res (Phila). 2012; 5(4):675-84). PG up-regulates stem cell markers DCLK1, CD44 and LGR5 (Sarkar et al. Gastroenterology. 2011 February; 140(2):583-595; Sarkar et al. Int J Cancer. 2012 Oct. 1; 131(7):E1088-99; Singh et al. Curr Colorectal Cancer Rep. 2012 December; 8(4):277-289. DCLK1 is required for proliferation of tumor cells, and protects survival of the cells. (Singh et al. Curr Colorectal Cancer Rep. 2012, 8(4):277-289; Sureban et al. Gastroenterology 2009; 13:649-59; Metcalfe and de Sauvage, Nat Genet. 2012, 45(1):7-9). DCLK1 has been recently proposed to be a specific stem cell marker for cancer cells (Ref 7,8). Long and short isoforms of DCLK1 are expressed by neuroprogenitor cells. (9) However, the relative role of the two isoforms in colon carcinogenesis remains unknown.

The inventors conclude that L and S isoforms of DCLK1 gene are transcribed by the use of alternate promoters 5′ and intron V, respectively, and differential regulation of the two forms indicates proliferation/differentiation of stem cells in normal versus cancer cells.

Expression of DCLK1-L and DCLK1-S in Human Colon Cancer Cells.

While majority of human colon cancer cells overexpressed the short isoform of DCLK1 as shown for HCT-116 cells, a few cell lines like HT-29 did not up-regulate the short isoform, which reflects differences in mutant phenotype of the cancer cells. A majority of the colon cancer cells down regulated the long isoform (like HCT-116 cells), but a few, like HT-29, continued to express the long isoform. A majority of human colon cancers switch on the expression of several autocrine growth factors, including progastrin. This progastrin elevation has been detected in various primary colon cancers and colon cancer cell lines, such as HCT-116 (Singh et al. Curr Colorectal Cancer Rep. 2012, 8(4):277-289; Singh et al. Cancer Res. 1996, 56(18):4111-5). A minority of colon cancers, however, switch on the expression of autocrine growth factors such as IGF-II, but not progastrin (an example is HT-29 cells) (Singh et al. Am J Physiol 1994, 267:G608-G617). Thus, PG expressing colon cancers are positive for short isoform of DCLK1 (Siddheshwar et al. Gut. 2001, 48(1):47-52), which can be used as a biomarker for colon cancers. This information combined with targeting cancers with PG antibodies/vaccine ±Annexin A2 Antibodies (for reasons described in Singh et al. Curr Colorectal Cancer Rep. 2012, 8(4):277-289; Siddheshwar et al. Gut. 2001, 48(1):47-52; Singh et al. Oncogene. 2007, 26(3):425-40) along with small molecules or shRNA against short DCLK1, will likely be most effective for treating PG expressing epithelial cancers and targeting cancer stem cells.

PG Expression can Increase the Expression of Short and Long DCLK1.

Progastrin expression Increases both DCLK1-L and DCLK1-S in HEK-mGAS Cells. But, Long iso-form is silenced in majority of colon cancers (FIG. 5), and thus PG expression is unable to up-regulate the expression of long isoform in situ in tumors silenced at the CpG site in the 5′ promoter of the long isoform (Analyzed by qRT-PCR)

Expression of Progastrin and DCLK1 by Normal Vs Colonic Adenomatous Tumors (Benign Polyps) from Patients, Obtained at the Time of Endoscopy Via Our Approved IRB Protocol.

Antibodies currently available in the market, do not differentiate between the long and short isoforms, since most have been generated by using extracellular domain segments from the C terminal end of the molecules, which shares significant homology between long and short isoforms. While one can develop specific antibodies against long isoform, which will not detect the short isoform, it will be a challenge to develop specific antibodies against the short isoform, as the short form has >90% homology with the long isoform, and lacks the N terminal end, as shown in FIG. 7. There are however unique sequences in the short isoform, which could be used to develop a specific Ab against short isoform.

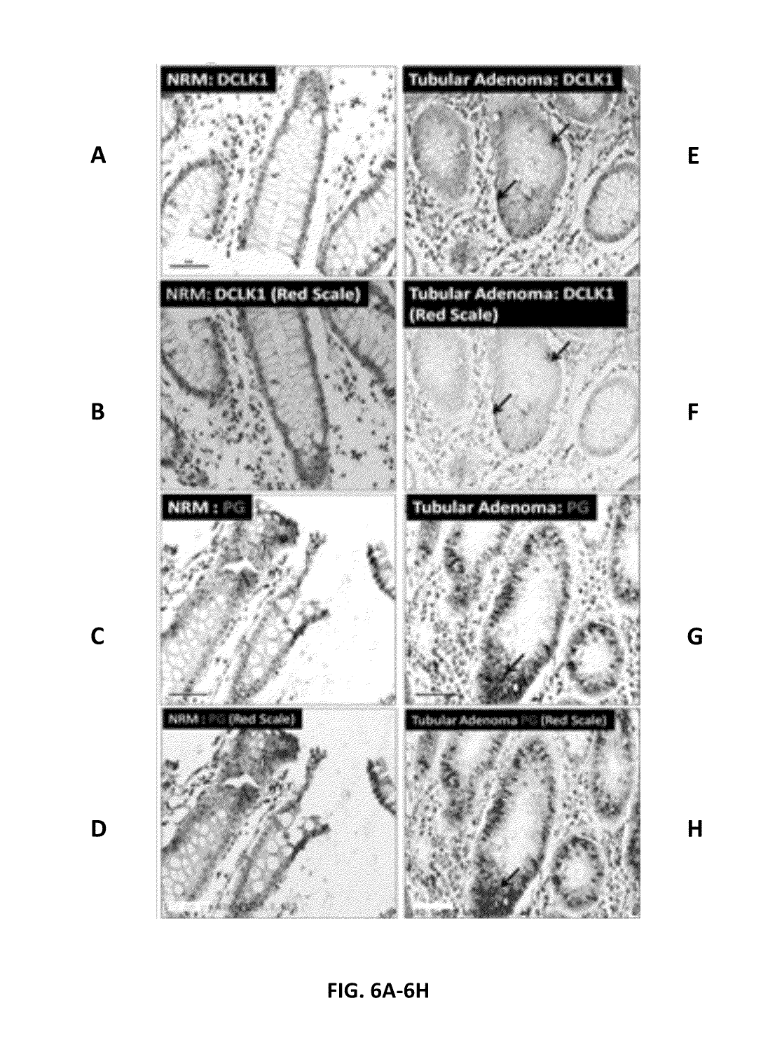

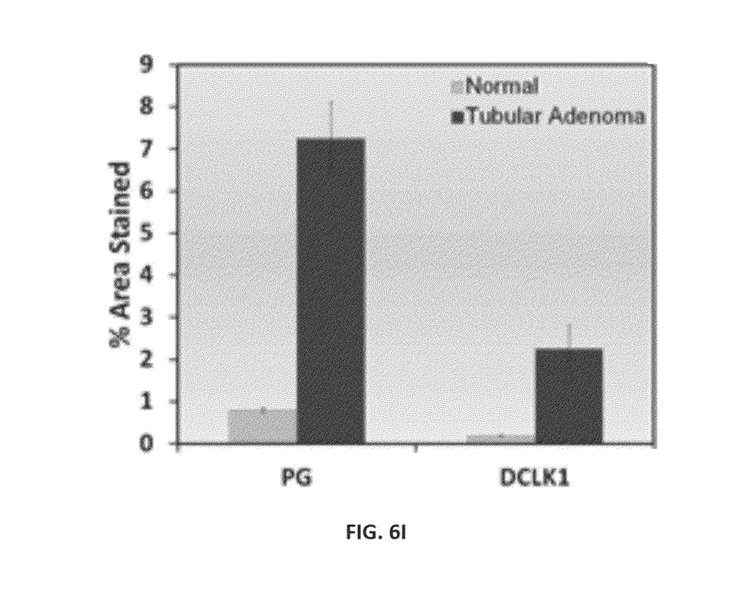

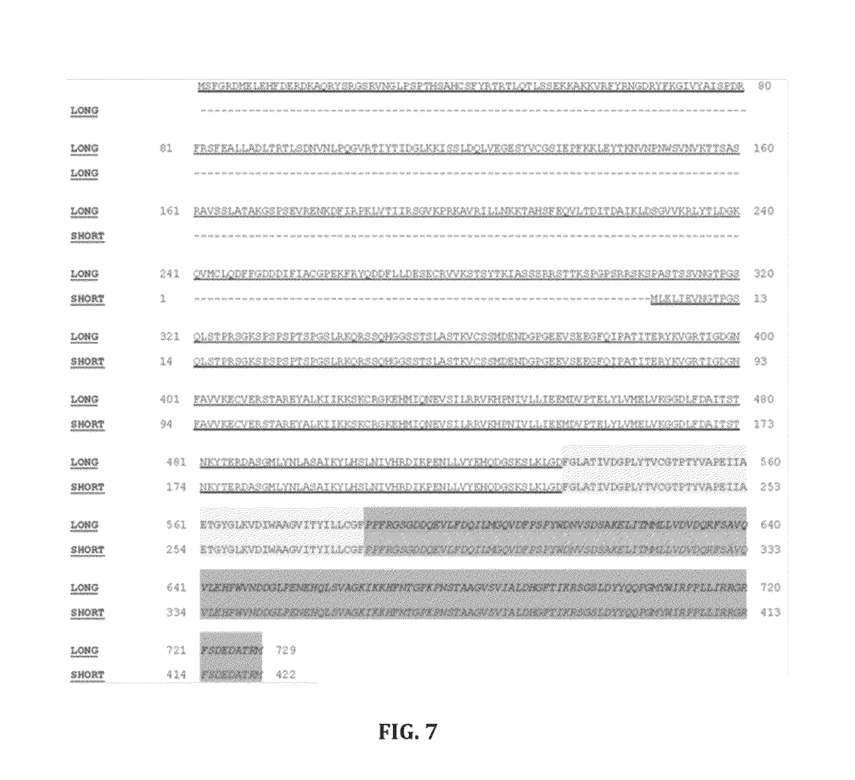

FIG. 6B shows human colonic tumors positive for PG expression demonstrates increased expression of DCLK1 protein even at adenoma stage.

Expression of Both DCLK1-L and S was Significantly Increased in Colonic Adenocarcinomas from Patients as Analyzed by qRT-PCR.

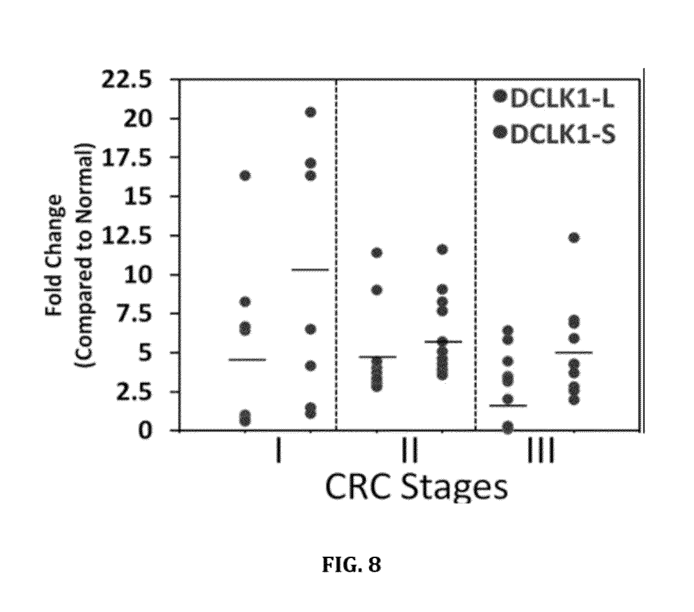

A cDNA plate was obtained from Origene, which contained cDNA from ˜40 patients with adenocarcinomas at different stages of the disease, and cDNA from 5 normal mucosa. The data are expressed as fold increase compared to the mean of normal values (FIG. 8). Normal tissues analyzed so far only express the long isoform and are negative for the expression of short isoform, which appears to be specific for transformed/tumor tissues. During the process of transformation, the long isoform appears to become increasingly silenced, and stage III cancers express relatively low levels of it. The presence of short isoform may be specific to transformed/cancer cells.

Thus, normal colonic stem cells appear to mainly express DCLK1-L isoform, which may be required to maintain stemness and proliferative potential of the cells. Tumorogenic transformation of cells results in the added expression of the S form, which may be dependent on autocrine growth factors such as PG. Therefore, the S-form can be diagnostic for transformation. Over expression of DCLK1-S in PG positive tumors may serve as a useful diagnostic/prognostic marker for colon cancer patients, and provide a useful target for treatment.

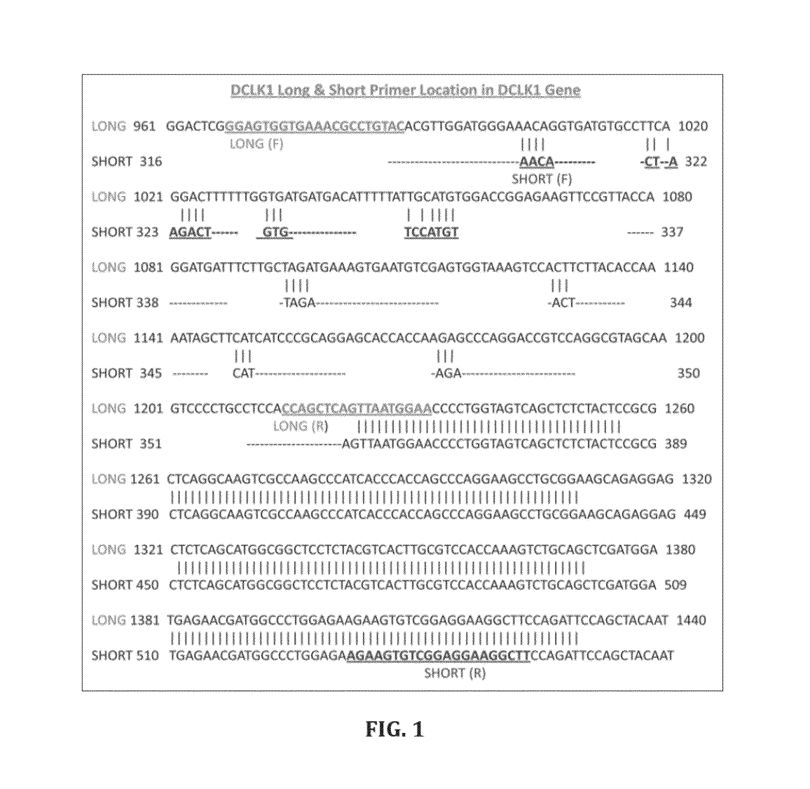

Primers were designed to amplify either the short (S) isoform, or the long (L) isoform, specifically, and confirmed by sequence analysis (FIG. 1).

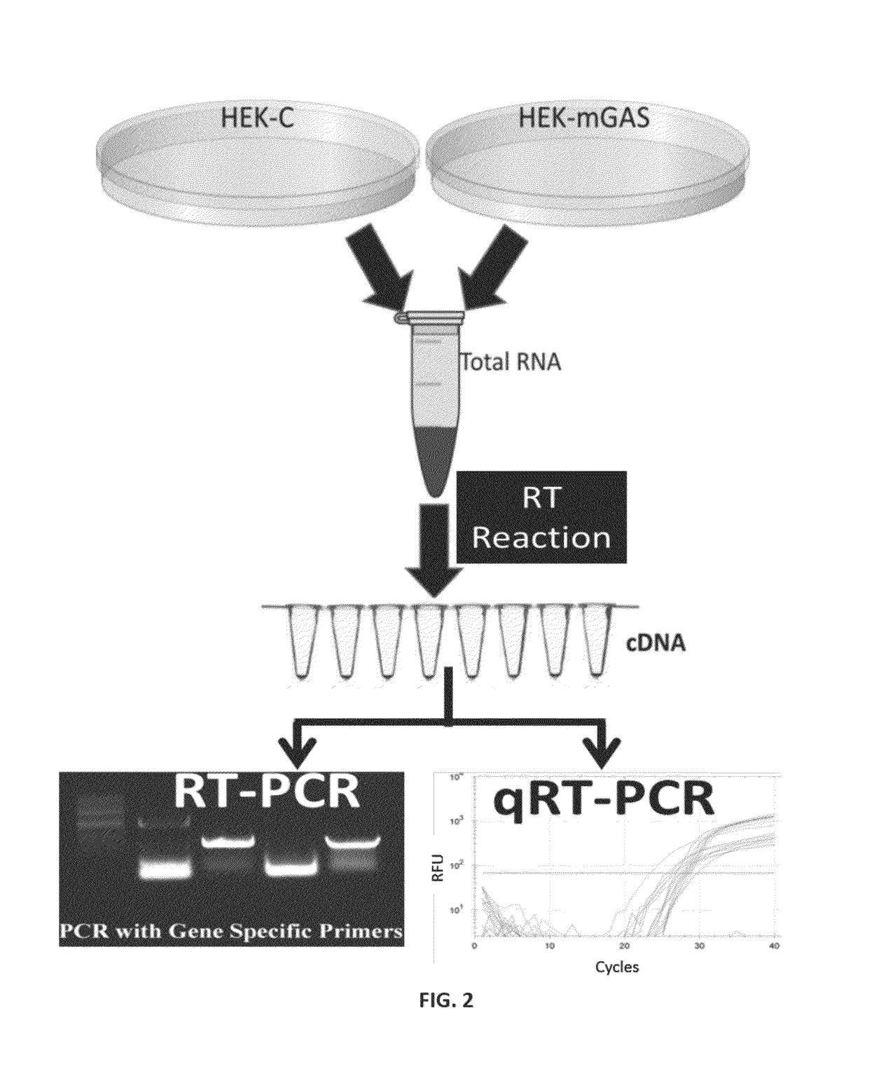

qRT-PCR conducted using, for example, the steps illustrated in FIG. 2.

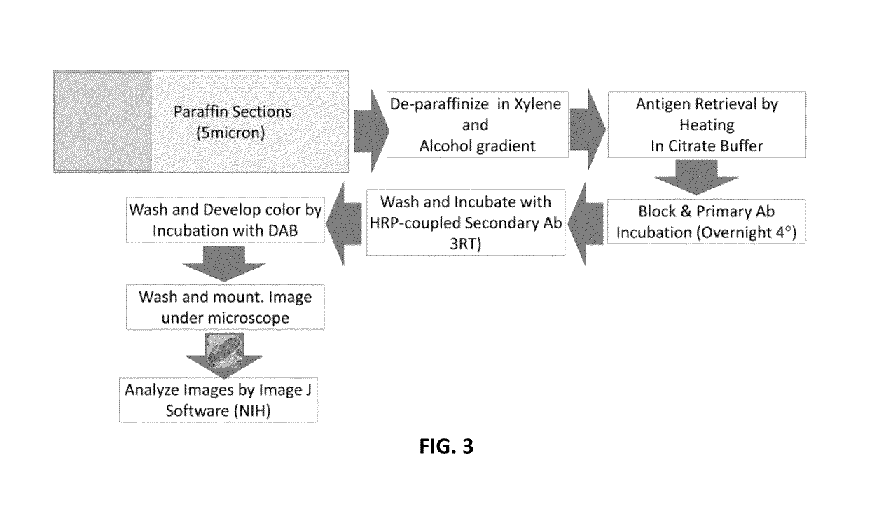

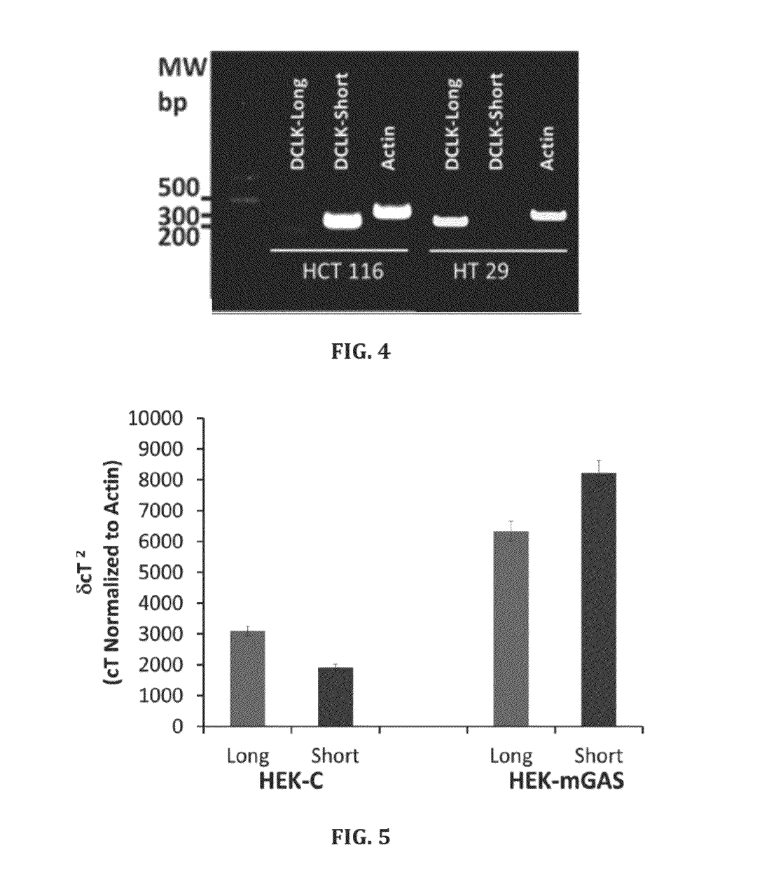

Immunohistochemistry of Tumor Tissues conducted using, for example, the steps illustrated in FIG. 3.

### Example 2

### Methods and Reagents Related to DCLK1

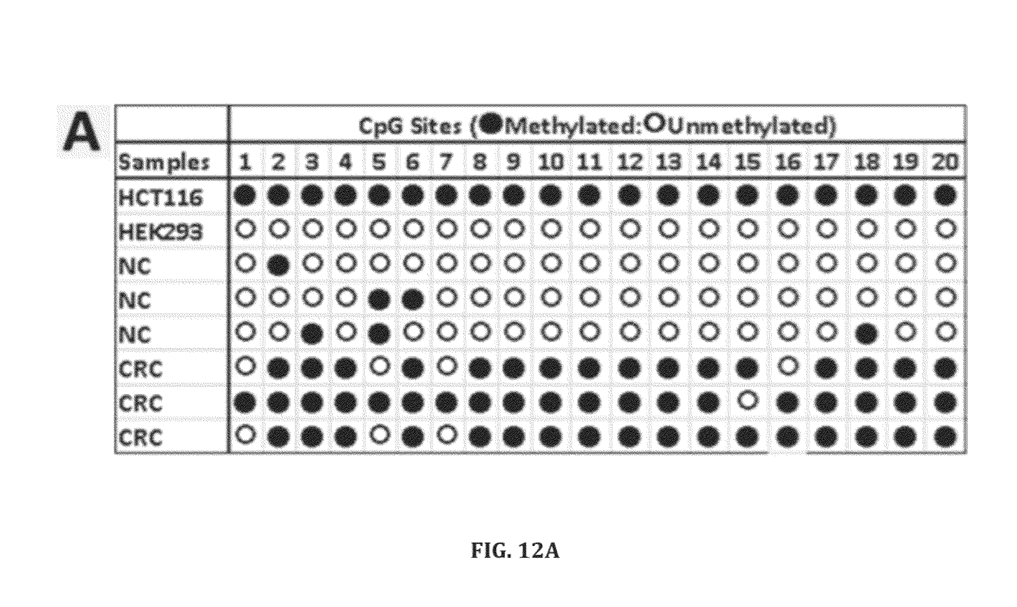

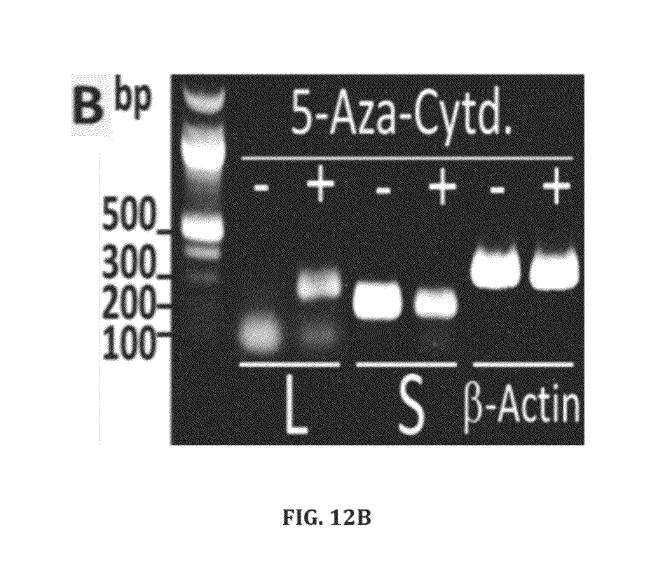

The inventors discovered that L isoform was poorly transcribed in CRC cells/tumors due to DNA methylation of 5′ promoter, as can be seen in a representative colon cancer cell line (HCT116 cells), while no methylation was evident in a non-transformed cell line (Hek 293 cells) (FIG. 12A). Treatment with 5 aza cytidine resulted in re-expression of L-DCLK1 by HCT-116 cells, confirming the silencing of the promoter by DNA methylation (FIG. 12B). Recent reports have confirmed DNA methylation of 5′ promoter of DCLK1 gene in CRCs (Vedeld et al. Epigenetics 2014, 9(3).30). Thus, DNA methylation of 5′ promoter leads to epigenetic silencing and loss of L expression in cancer cells/tumors.

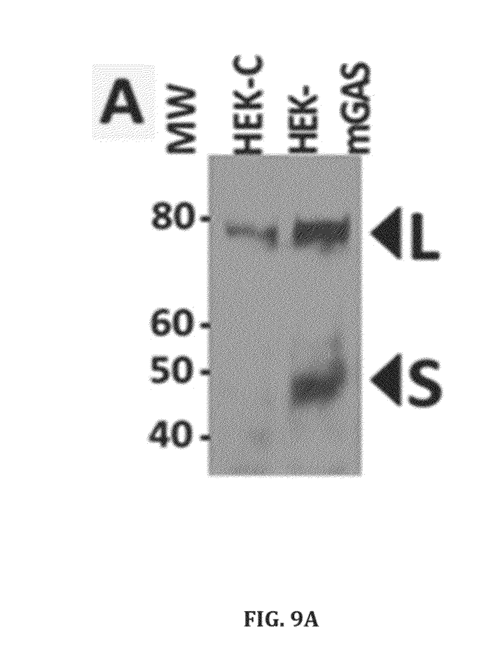

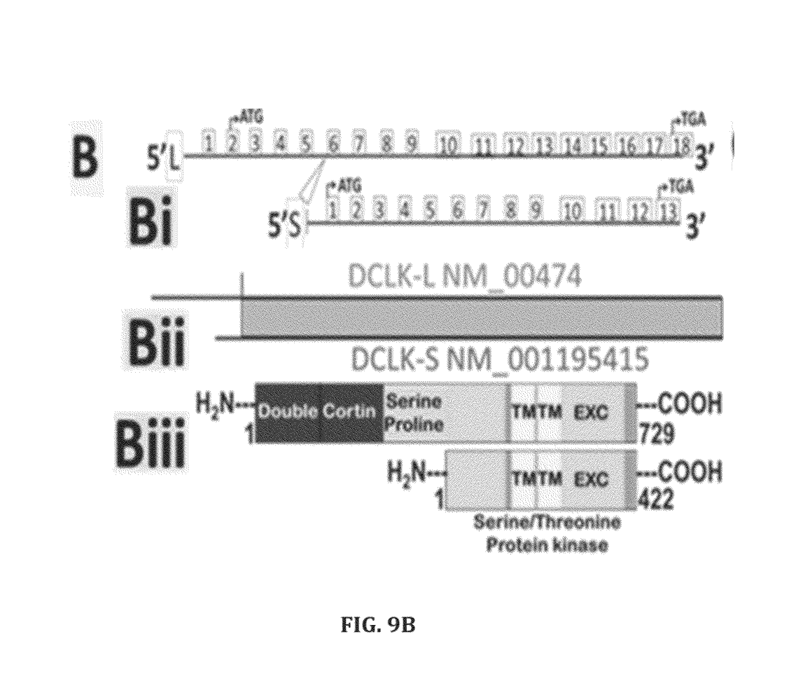

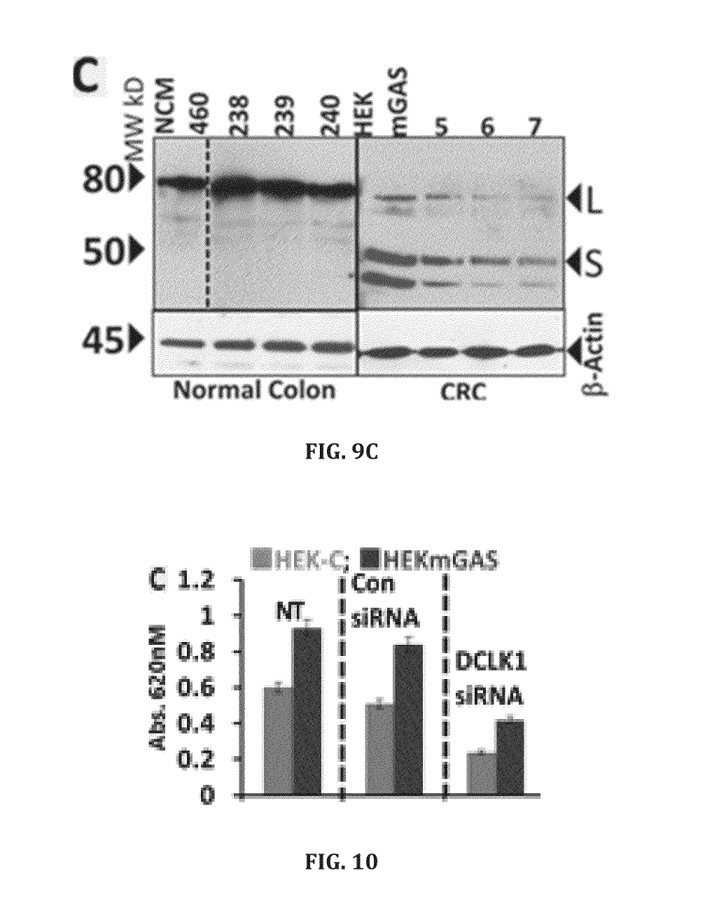

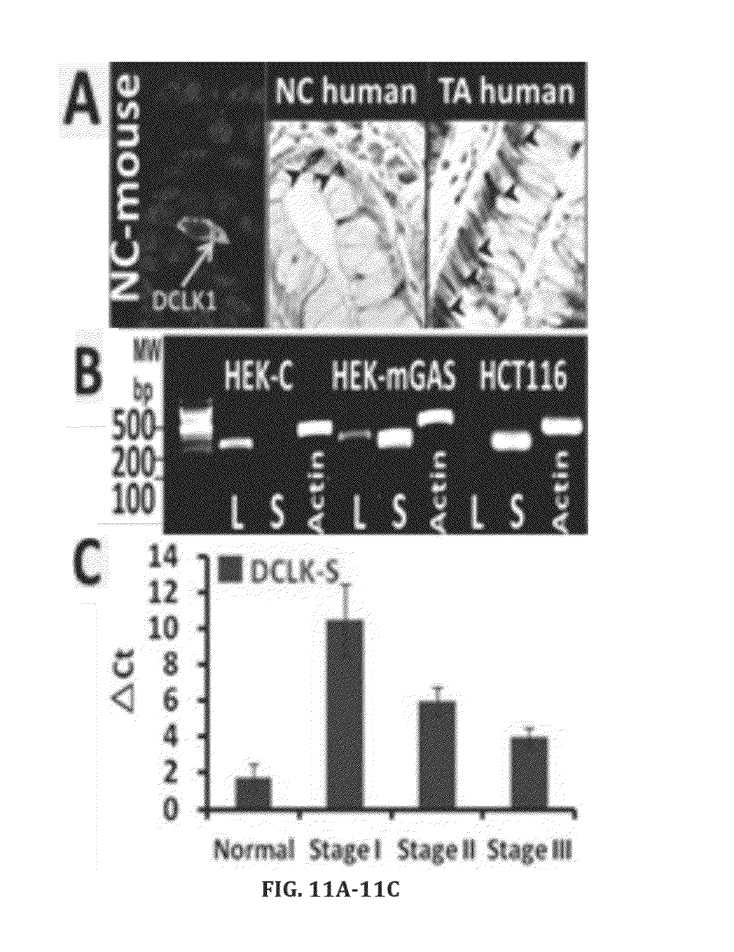

Studies with isogenic HEK clones demonstrates that tumorigenic transformation (HEKmGAS) results in up-regulation of S-DCLK1 (FIG. 9A). DCLK1-siRNA, which down regulates both forms, attenuates growth of CRC cells in vitro and in vivo; therefore specific down-regulation of DCLK1-S, without any effects on DCLK1-L, can eliminate colonic CSCs, while sparing normal stem cells (NSCs). Since HCT-116 only expresses DCLK1-S, due to epigenetic silencing of 5′ promoter (L transcript) (FIGS. 12A and 12B), isogenic clones of HCT-116 were generated which either expressed control shRNA (ShC clones) or DCLK1 targeting shRNA (ShD clones).

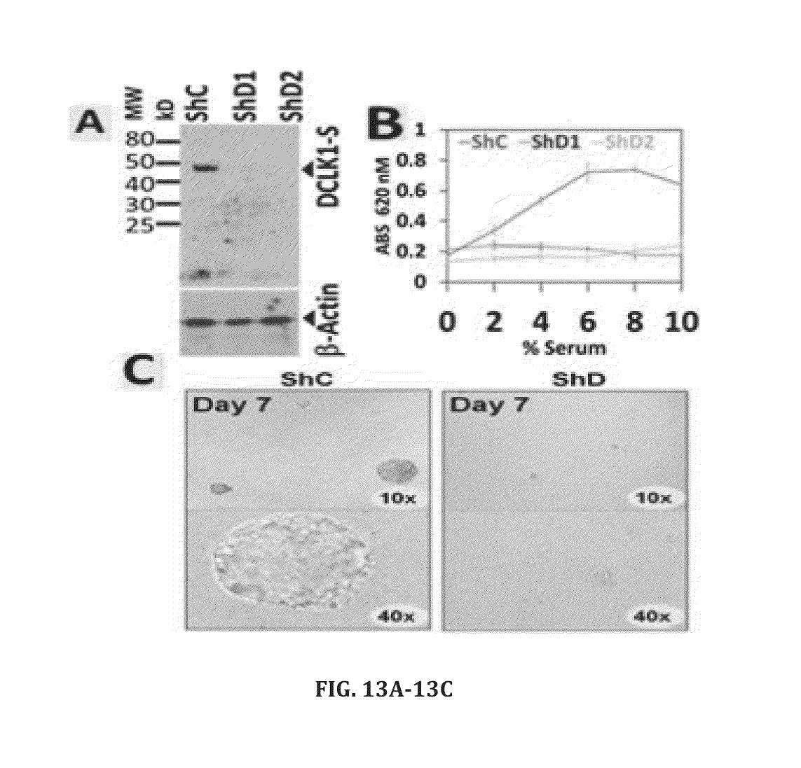

Western blot (WB) confirmed DCLK1-S attenuation in ShD clones (FIG. 13A). Proliferation (FIG. 13B) and clonogenic potential (FIG. 13C) of ShD clones was significantly attenuated compared to ShC clones (FIG. 13B), confirming that S isoform is required for maintaining proliferative/tumorigenic potential of CRC cells.

ShC and ShD (KO) clones were subjected to RNAseq analysis using non-biased next generation sequencing (NGS) in the Core Facility at UTMB. Genes down-regulated by >3-7 fold in ShD vs ShC clones were validated at RNA level (data with a representative gene, Col3A1, is presented in FIG. 14A). The major hypothesis tested was that S isoform supports tumorigenic potential of CSCs by up-regulating a unique set of genes, while L isoform in NSCs regulates a separate set of genes for maintaining multi-potency of NSCs. This hypothesis was confirmed based on the results of the RNAseq data. Genes that were down-regulated in ShD clones of CRC cells were determined to be dependent on S-DCLK1, while their expression remained unchanged in ShD clones of non-transformed cells, that were only expressing L-isoform.

The inventors developed bioassays for high throughput sequencing (HTS), using promoter-reporter constructs of genes identified as being down stream of DCLK1-S, but not DCLK1-L (FIG. 15). As an example of the bioassays developed, the inventors generated promoter-reporter constructs of COL3A1 gene in pCLuc-BASIC-2 vector, based on published sequence of the Col3A1 gene (FIG. 14A-14C) and transiently transfected ShC/ShD clones of HCT-116 cells, as per protocol shown in FIG. 14D. The ShC clones were confirmed to secrete >5-fold higher levels of luciferase in conditioned medium (CM), compared to ShD clones (FIG. 14E), demonstrating development of a robust bioassay. Similar bioassays have been developed for all genes indicated in FIG. 15. Data obtained from the HTS assays with the specific bioassays will help identify specific molecules from available chemical libraries, which will then allow the development of safe and effective drugs for inhibiting tumorigenic/metastatic potential of cancer stem cells, while sparing the functions of normal stem cells in intestinal crypts, as a non-toxic treatment strategy for colorectal/pancreatic cancers, and other cancers as well, which are found to be dependent on the expression of DCLK1-S.

Bioassays for High Throughput Screening (HTS).

Promoter fragments of ˜1-2Kd, of the identified genes, downstream of DCLK1-S (FIG. 15) were PCR amplified from gDNA of HCT-116 cells and cloned into pCLuc-BASIC-2 Vector. An advantage of this plasmid is that cypridina luciferase is a secreted protein, and thus the supernatant of the transfected cells is used for measuring luciferase, without lysing the cells, as shown in FIG. 14D, for a representative gene. The minimum promoter, required to measure maximum transcriptional activation of the gene in ShC clones of CRC cells, was used for generating stably-expressing clones of HCT-116 cells (named Test Clones for the specific gene). DCLK1-siRNA treatment of Test Clones results in loss of luciferase activity by >5-7fold, compared to control vector levels, confirming the validity of the developed bioassays (an example is shown for one of the genes in FIG. 14E).

Loss of luciferase activity in conditioned medium of Test Clones is being used as a readout of inhibitory efficacy against functions of S isoform. Initial filtering of the compounds will be done in HCT-116 cells stably expressing cypridina luciferase, down-stream of CMV promoter. This filtering step will eliminate non-specific inhibition due to non-specific inhibition of transcriptional activation of reporter constructs or luciferase enzyme. These experiments will help us to discover lead compounds for developing inhibitory drugs for specifically targeting CSCs, while sparing NSCs, for all cancers dependent on the expression of DCLK1-S.

Bioassays with Promoter-Reporter Constructs for the Two Isoforms of the DCLK1 Gene.

The 5′ DCLK1-L promoter-reporter construct and the DCLK1-S intron V promoter-reporter constructs that have been made and cloned into cancer cells for purposes of conducting HTS bioassays are shown in FIGS. 16A and 16B. These tools will allow us to discover specific inhibitors that reduce the activation of intron V promoter (and thus the expression of DCLK1-S) without any effects on DCLK1-L.

The primers which were used for amplifying the S and L isoforms are shown below. Since the DCLK1-S is >98% homologous with the L isoform, the forward primer of DCLK1-S as shown in the figure was the only one of several primers made that could amplify the S isoform, without amplifying the L isoform.

| Primer Name | Species | Sequence | PCR |
|---|---|---|---|
| DCLK1-Long | Human | Forward: GGAGTGGTGAAACGCCTGTAC. | RT-PCR & qRT-PCR |
| | Human | Reverse: GGTTCCATTAACTGAGCTGG | RT-PCR & qRT-PCR |
| DCLK1-Short | Human | Forward: ACACTAAGACTGTGTCCATGT | RT-PCR & qRT-PCR |
| | | TAGAACTC | |
| | | Reverse: AAGCCTTCCTCCGACACTTCT | RT-PCR & qRT-PCR |

1. A recombinant nucleic acid comprising an expression cassette comprising a DCLK1-L promoter having a nucleic acid sequence of SEQ ID NO:1 operatively coupled to a reporter gene.

2. The recombinant nucleic acid of claim 1, wherein the promoter has a nucleic acid sequence of nucleotide 1202 to 4000 of SEQ ID NO:1.

3. The recombinant nucleic acid of claim 1, wherein the promoter has a nucleic acid sequence of nucleotide 2277 to 4000 of SEQ ID NO:1.

4. The recombinant nucleic acid of claim 1, wherein the promoter has a nucleic acid sequence of nucleotide 3109 to 4000 of SEQ ID NO:1.

5. The recombinant nucleic acid of claim 1, wherein the reporter gene encodes a luciferase protein.

6. A cell comprising the recombinant nucleic acid of claim 1.

7. A recombinant nucleic acid comprising an expression cassette having a DCLK1-S intron V promoter having a nucleic acid sequence of SEQ ID NO:2 operatively coupled to a reporter gene.

8. The recombinant nucleic acid of claim 7, wherein the promoter has a nucleic acid sequence of nucleotide 1497 to 3604 of SEQ ID NO:2.

9. The recombinant nucleic acid of claim 7, wherein the promoter has a nucleic acid sequence of nucleotide 1497 to 3229 of SEQ ID NO:2.

10. The recombinant nucleic acid of claim 7, wherein the promoter has a nucleic acid sequence of nucleotide 2652 to 3604 of SEQ ID NO:2.

11. The recombinant nucleic acid of claim 7, wherein the promoter has a nucleic acid sequence of nucleotide 2652 to 3229 of SEQ ID NO:2.

12. The recombinant nucleic acid of claim 7, wherein the reporter gene encodes a luciferase or eGFP protein.

13. A cell comprising the recombinant nucleic acid of claim 7.

14. (canceled)

15. A recombinant nucleic acid comprising an expression cassette comprising a COL3A1 promoter (SEQ ID NO:3), ZEB2 promoter (SEQ ID NO:4), NFATC2 promoter (SEQ ID NO:5), TNFRS6B promoter (SEQ ID NO:6), or SPARC promoter (SEQ ID NO:7), operatively coupled to a reporter gene.

16. A cell comprising the recombinant nucleic acid of claim 15.

17-26. (canceled)

27. A method for identifying a specific S-DCLK1 inhibitor comprising;
(a) contacting a cell comprising a S-DCLK1 reporter construct with a candidate compound;
(b) assaying the cell for expression of the reporter construct; and
(c) identifying a compound as an inhibitor of S-DCLK1 if the expression of the reporter construction is significantly decreased as compared to control cell comprising a S-DCLK1 reporter construct that is not exposed to the candidate compound.

28-34. (canceled)


SEQUENCE LISTING (16 sequences)

SEQ ID NO: 1 - DNA, 4264 bp, homo sapiens
tccaaaaggatctcactcaacaggtttcaatgggggagcaaataacaatacgcaaggttaataaaggaaaattccaagtgggcactggacttaggtcccccaaatctaattaaacaaatgcagaatgagtggtcttaagagcaaaacatgtgaaaagcatggagatttttattgactggatgagtcaatagcatggcatgcctgtcagagacattgattagacgttagcttcctataatggaagcaccgtgtcaacagccaatgaagtcatggagcttccctagtcagaccacccctgctgtgccatgcccagttccgggaggcagatgtggagggagacactgacagttgcactgctgtgagcatttccaaagatgtcaaatgagaagcagttgaagggctgaggattcctagttaatgaggagtcaaatttgggagaaagtaatgagactgtcaagcaaaagccaggaccatttgatttgttctccacaggtctaaagggtagacccagggtacacaggaaaattccagggaagtggatcttagttgaatttgaggagctgtccagcaggggagtgggctgtgcagagttcccctttactaaagcaaattacatttaggcaggaaacgaagaaaagagctttcagaagctctctgctagtttatagaggttctatgagagtagtaggagcctttcttcctctgtgtaggtccagaatctcatgtagtttggacctcagcatgtttggagggccaaaccaaaggtaaacagccagttaaaacaattatttttaaagatctcatttactgtgtctagaattccatgatctatcctaacactcaaagggataatcttattcctcttcacttttgccacagacgcaacagaatggaggctggaagacaacttgttgtggagggaattcatgcactgggtaaaaattgataggcttctgccaaggtctgacatcaccgagtttctgtgattctagtagaagccagaagtatgttctctgtaattagagggaattagacaaactatcaggaaatgccatacagagccttcagaagaaaagaaatacaaaggaaaatcagactgacgattatgttctgtaaagctgcttctcacaattgtggcagccactacatatacacagtctcccaacccaggagcactatatgcccatagccacagaggagagattatgaatgagccagagcaaattcttcctcctagagggaaaaacaggaagtgtatctcttcctgctcatattctcattctacagttctactcgcatctacatgattaagcatattgtttatgaagaagccaatgaaaagaacctgaaaaaagatacatggtgaattataaagtattattccgaagaataatggttctctgtctctttcctggtttgcactaaaaccttacattcaggaagacaagcacacttagataagattatccatcctcttcgttgccagaggttaaagtgcaaatgtcctcatgagaagtttagtccaaagacacatttgaggggatccatcactaaaagaagcagtgttcatgatagcaaggagttaactctctgttatgaagtaataagctctttgattaaaagactgcattttccaaaaaagattacactttgcactagaatttggtattgctttataagcatatggccttagcaattgaaaaactgacttgaattttaaaacataccgattttaaatattgaagttgggcttgacatttttaagctacatgactgtgggcaaatgatgtaacctctctatgtctaaattccttcatctgtaagatgaggataataatgccaatgcctaccttgcatggttcttatgagaatgaagtaagaaaatgtatgaaagcccccagcatggcaaaaagcaagcactaaattaatgggagccactgagagagttgaggcctagtcattttcaacctgtaggatatagagctgtgtctgcttggggcggtagttggggtacagcttaaatagcatttgcttttgggagctagaatgagatctccaatactttcagatgatcctgggggaaaggatgtattagttccattcacttctttgatcaatatcttagtaatataaaggaagtagcattcgtttttgaaaattacttgaattaggagttttctagttagatttcatttcaataaggaattattgttgaatataaattagatttgccaaatgcttaagctgccctggaatgaactttagtaccagcctgctgtcctggactaaatgatatgaccatactgtagggccaatgtatactatttaatgtcttcatctacttaattatcagttgccccctggtttccttaaagagctaatgatgcccaactatttctagtgagagcactttgctactgagagagtcaaacaccaaggtgattctctgaacacattatctgtgtgtgctaggtacaacagctaggaagaggtaagatgatggtgagttacttaaagctcttaagaaatgaccctagtagaatatccataggggttaagagatgaaatttatttcttggcttagcattttggaagaaatcacttggcatttgtcattggcaagataaaaagaagctctgatttctgttctccagaagatgtacttggcttcttcctttctcttaatttacccctatggctctcattttgcctcagaatgtgaaagaataggcttactactttgcatttagttaatttggtgagttcactgtactctcatctctgggctattaaatagttcaaaaataaatatcgcatcacttgtggaatatgtagcatggatctcatttgagctgtgttttaaaacaaatggtaattatttaacatttacatcatacttggaaagtatttcagaatagaactttttaataataagtagtcaaacacataactgggatcaatgggcaagagaaaaataaaattttaatgttccattgaaaggtgtaggctcaggaagagagaaaaatacttatgtgtattttgtatactcatgtgtatttgggacaaacacaggttgtggacgacaggattgaaagtcagattaactggatcaaaattcaggctgcgtactgagccagtctaagatttcaccgaaatctgggagctgctcccactccctggacttttgtgcaaagactggaggaagctgtgaacagcctgggctaggagaacattccccctctgggaggagaggctcgcatgagctcatggggctttccaccgcagacccagccttggcagccacaaagtgtccagcagtgaaacctggcccttcagttctcaagggccctttggaacatatttgactctaagcagaggtcactattccaagagtgactcatgtcttggggttaagtggagatgatgggtgggatccatgaacagatccagctcttcccaatgtggggggcaccagagtgcatagcttgggagggttggtcatccgaagaggcactgcgtgggtgcatcccgggcaaaaaggatgagaaggtgatccactggcttccataccctgggaaaggtgtcagaccgtgaggtcacatcaaaaggtatgagtgtgggtacctccttccctcccccgacggtgggggcctcatctctcggccatatcacctctgtgccttgcacacttcctggctcagactcagccctgtccacgttctcttccaccatctgatctgcccccatctcccaaagactacttgtggggactagggaggtttaggggtgtagtcaagccagatgcaccgaggttgctgcggtgggaagagggcgctgggagggaggaggacctggggcgcagccgtggtgggtgcgccctgccaagaagggcgggggcgacggggcgcgcacgcggaggaggaggaggaggagggagacgggagggcgtgtgagcgagtgagacaagaaaagggagcgcgcccgccgccgccgccgccgccgccctcctctggagagagaggctggagtgaggctgtgcgaagcgccgcatttcaatgaggacgggccgaggcacatccctgcactagtggccgcaaccgaggcgccgcgctccagcagctgctgccgcccagcccggccccgccgccgccccccagccctgcagccccgcagccccggccgcgcccagcccggcgaggacagcaccaggaggcggcccccagcgcggccacaaagacccccggcggcgtctctccgcggaccg

SEQ ID NO: 2 - DNA, 4352 bp, homo sapiens
ttaaaagggcaatgaatttgtaaattaagccaagtatttccaaattctagattttttaaagtaactttgatgtttaatcataagcatcttggggacagggagcaataagaatctagcatgtactaagtgtacactacgtgtgctgataggttgatatctagggccctggactcgtggctccagttagtgaagaagagttggttcacctgtctgaaatgatgaaggaaaaggtggatctgcacacttcctgaggagtattattcaacaatgcgctgtgaccggtatatgctgttggatccctgacaggcttaggtgacagctgagtcgaaataaaggagggtcatgtaccgtcgggagggatagaagtcagctctgcagtgagatttcttccacagctctttggtggccagtgatgttaattggtcatcatggtatcagtgaatgatccattttgaagttgagcacttctcattcaagggtaacaagcacagcaaaggatgacatttcctattttttaaaataagaaaatgtacctatacagataaaagcaaaggagaccagaactgggtaaaggcagcggttctaatgagttctttactaattcagagtttagtggattcagattgaaataattaattgttcagccctgcttggctgtttaacaacaaccgtgttagtttaattgttcccattcaagataaagaattgtgtaataagggcctctgaaaaaataatgccattttaagttgaattttaataagttctattaaataagtcacaaagccttgttatggcacttagaaacttctggcattacagagtaaaaaccaagtgtactcagtacatgtgtcaaacttatttatgaaagaaaattatttatcccagcagtgattcaacaagaaggtgtgtaatgctttctggatgtcaagaaccattttaagaagttgctaatgctcttctgtatggccaagaggaagagtctgaacagagctctgaccctaagagtacatgctgttcccaatgttcagacaagttgtgttccaaaagcatttgattgtaaatgacttgtctggcacttgagtttcatttttctagagatggcgatgcaaatagaagcactgagcccagtggttttattaggttggtgcaaaagtaattgcagtttttgctttacttttaatggcaaaaaccacaattaccattgcaataaccttgcctaaaggaggcagatccgagtgtcccggtggtagagagagaagcctaggcatcctcagccctgcagatgctaggagggttctggacatgccctgaggtgccaggaccagccactcatgggaaaagtgcaaagttttttgagtaagacctaaatgaaatccagctttaactctgccatcttccagctgtttgtctttggacaagttacctgactaaacatgtttcttcatctaaaacagaaaacattgcaccaacttacatggcagaactgatgttacctaaggtgcttccgttcaaagtgtccaaactctctgtccttgcaaacatgctcccttttggccttaatcttctacagccaatcaattaccaggttctatagtttttacctcttacatttctctggaacgtttccgtttacctccaccccatgacactaccctagatcagccacgtcagcatctctaccataggacccttaaatgtgccccactctctcctaccttgacattctccttgcacccagtattgtttccctctaatccattcttgacagggcaagagctggagagcattttcaaatgcaagcctgatcctgtggcgcctgcttaaaatctttgtttttgtttttgtttttcttctcattgccctttggataaaaaccaaactccctaacatgaattttaaagcactactcactatgcggcccctgcctccatctcattcctgacattcctccgcctccaaccccacatacttgctttacttagttatttaagtctcttcagtgtaccacactggcctcctttctcgtatccagccctgaaacaggagatccctgggctgaggacacttttccttcattgtgccccccttgcttcaccaggctaacttctgtatccactgccctctgtgaagggcgtaggccaactctcactgttggttattaggattccaattgcaacacccaatacttctgttaatggctgttgtgatacctcggttcttgtcttcttagtttaaaagaatttaaacaaaagacacacagcaaaggagatgcggcagagagtcattcactgcaaaaggaaaagagtattttgaaagttagatgcagaatagacagtataccctgagagagagagttcagggtgggctgcttgcaagggtgaggcagtgttgattattgctagagaaaccccctttgtgggagtcttacatgataattcataaggagttgggaagaggtgttgctagtaagcatgttctgggtggtcctctgggtgcacatgcactgtagctgtatgtttgtgttcatatatcacatgtctcattagcatcttaaatctccacccaggggtgtgttttttactataaatgagcaaagggtcagtttgatgacaattaaaatcaaaatgcacatgccctagagaagggacagtccctcctgaagatagctttgcttaaatgagctcatttatgatgtgaatgccaaggcttattgtgttggctgtgaggtcaccaccaatgctgcatcccgagaacatggtcacttccttgactaactatcctgcctcacttcccctctcaaaatactttattgtaattctagcttagttctctggtttacctgtaggttaaagggctggcacagtaaggcaaggtagtagtaagactgcaggttttgaactgcctaggttagattttaggctctgctccatatttgcttgtgaaatgactcatcatctgtaagcctcaatttcctcacctgtaaaatgggtggaaatgatgctgccaatcttatagagttattctgtgagttaaatgatgtaatatatatacatactgatgcacacagatgcatatatgttcatatataaaaggtctaagtgcattgactggtgcataaaaatgccttcaataaaaataaatgtctttcaaaaaatgccttaaaaatacacatacaatggatacttactaaatacttacaggcttttattgttttgttttgctttttaaagcaaggtattcctgagactgttttcttgtgagccctttgtctctttttcttcattttccattagctggataagatcagagtctcaaagtgaaaagtgttaacactgtgtttgcttccctatttgctcattttttccctccaatctatgtagcctttacgtatgacataaagatgtgcagtagaatgcgggtgggtaaagaaaatattatattcacaaaatgtgttctccagccaaccagtaaggcatccctttcttactagttataggctctgcttctgcccttgtctttaatccttttctatttctgagccccttccttagaggctactggctgaggaagtctaatgtgatgaggtccgagcgtgaagttcactcagcaaagacaggatggtggcatgcaagcaaattaaagcattctcagctttccagttcactctctctgcaaacaaacaaacaaaaattcatagctttttaggtttctatttttaggagttgccaaatacatttcaaaagcaaaactacaaattaatgcagcatggggtatgtgacattttttaaggtctttggaggagatgacaaaacaaggagcttcccactttccagggttgtaagtttttgggaaaacatcaagtgcgaaaaatcaataaacatcctaccttgccactgaattgtgtgagggtttcctgattacctggtagtcaccaggctttagaagagttattgtcatttcgattttcagatgagtcactggatctcttgcccccagggccacttcattatgattcagtgcaaagtactgacagcactggggcttccggaagggagtggtgtcgggatgggggttgctgttggcaagaggcttaattgacacctttgatgtagcctaagacagaacctgcacctcccactgctgcttgggagacgtcagctttacgcagaaaaggcatctgctgatgaatcctgcatctcattcagcctgctctgggagcagccttagccatccttaaactcatccaaccgattcccatttccgcgggtgcttggcttagtgctgagacgggattccgccgttggtattcaggtccaacactaagactgtgtccatgttagaactcatagaag

SEQ ID NO: 3 - DNA, 3054 bp, homo sapiens
  misc_feature (2893)..(2893): n is a, c, g, or t
gtagaaagcgctgctggcattcactggcctactgagttcatcacagctgtgcttgtgcatctctctcaagctccactttccactgaattggaaaagatttgaatttttggttgataatctgccacacccttgctttgcctcatctttccaggcactctctttgttaaaggagatccagattaatcagtgctttagttggtgcaggtatttagatttgtgttaaagctccaaatgcaatccaaaaagcaaaatgacatgtttatgtgtcctggttaacatcagtgaagtcagggaagtatgggaagaaatgtgtggctagataacaatgaggacagcagttcctggatggcataataggattcagaagtgtatgcatttcacctagttcagaccctgtattaagaaatgctttgaccccgtattaggaaatgctttgacaggaggtagaaaaataggatctgggacacagaatgcaaaggatcctagtgcaaaacctccatattcgaggtagtgaagaatcgtaaatcagaatatctggagaatgaattctctaggtctctcctgaagaccttaaaatacaacaatccttccatatgcaaaacatcagctaatgtttcttaagtgagtgcatcaatcatgaatttttctcatgagtataaggcttttatttgaatagctaagcaaattaagaattttagaacaatagtaattttatttataatgcctaaaataaagcataacaagcattattttaaaaataacatttctaatttttcacagtggaaaataaatctaatgaagaaattcaataaatttcagcttttatagaccacctaaaacaatgagagaaatctaatatagatatatgaattctacgtagatacaaagacatcatgagaaaaactatgtttctgaattggtttgaacacaggaaatattttttgtgtacaggacaaagatgtctgaaagacagaaagatgcatataacagcctttccccagcatctctactgtgatccagtagaaaagcttatattcatgaccattctcaacagttacttttctcccacgcccccaagcagtgactctccaatctaaaataaagatacacaagaggttggtctcgttctagttttcctttcctgtcatcaaatgatgtcatctacattgagatgtcttggtttgtgtccttgtgactgaagatataagaaagcttgggaggccgaggcgggcggatcacgaggtcaggacatcaagaccatcctgactaacacggtgaaaccccgtctctactaaaaatacaaaaaattaaccaggcgtagtggcgggcgcctgtagtcccagctactcgggaggctgaggcaggagaatggcgtgaacccgggaggcggagcttgccgtgagccgagattgcgccactgcagtctagcctgggcgagagagggcgaccccgtcgcaaaaaaaaaaaaaaaaaagatgcaagaaacaagaaagcaagtttgccactgtccatgcttacaggattcatgtacacctgggaatatacatatgacatgagttgagtgaaaatttagtaacacaaattagaagaaagagaaagatgaaaattagtttaaaggaggtaatttacttaaggaggaaagttttgcattgaggcataaggaagtgacttccattaattggtgtgaaatggggagaattttatgtgcacaggcatagagacggacatgtttaggtgaaggtgaagaaaattagttcaagtgaccttgattagaattgaaatgtccaccgtagattatagagatagatgaactaactatgaactgatgattagggtgtaaagagatatctccattgccagatatagccttatactgtggacaagcatatttttcctgcagagatttatcaaagacctataaatggtatttgcccctctttcaaatatgacacaaaaattactgcttagatagatccagaaatacattggaaagttttgtggatgaggatctggaaagtatacacataaaagtctaaattagaaatatttatactaatctttagagtaatgcagaaaaaatgaccactttctctggtactcttttgctattctatgtgataaactctttctcttccagtttcctaaaaagttatgtcatgaaaaggctgaatttttctctagaaaaatctgctctgtctttcccaggcagcataaaaattggaaatctcacaagaaagcacctctggcttgctaaagaaatttgtgagaaatggaaacagctatagataagtatctaacttttaggaagccattcaaacattgctgaaatactttgtcttttgatatttgcctgaaacttaacttctaggacccagggtgggtggatgagtcgaaggggcatacggtggcatttctttccgtgagtctcttacagtctcctatttaaattgagttaggattacttctggcaaaatcccaacataaaaatcttctaggaagatcagttctgtaaattagacatactagataaatgggcatcaagcagtttttcaaaattatgcagttgttaacttcataaggggaaataaaaatgtatgcatttacattgtatgtattaaaacaaggcagagcatttctatacgttcctaagttatacaaacatatatgtaagagtgaaatatgtaaaaaaacttttacataagcagatgcatacaaactccagatgtgctctttttcttactgtgggttgtgtcttctataagggaaaaagaaatatttatcatttcttttactgctgaggggatgggtgcggctctcatatttcagaaaggggctggaaagtgagggaagccaaactttttcctatttaaggccaaagcaaaggaatctcmrnaagtggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcg

SEQ ID NO: 4 - DNA, 3933 bp, homo sapiens
ctgattttttttttttttaatagactttcccctcctatcctaggctcaactccgaatggattggattgcgagtctgcacgtgagaaaaccgtttggcttggcttggacccctgccgccccccacctcctccacacacacccagtccaggggtcccctttatcaccctttgcttgcaactccaaaagaagttgcccacctcctgagtcacaacacaaggtcgaataattcctctagatgaaagatcagtttcatttcaaaacgagaataggttcctttttttattttctccacatggtacaaaataaacagaatttgctttaaaaaaaatcattagctgtggccaaattcgaattccttctacagcctgtgtgttcaagggcagaaacattgtatctcttcgggcatctgggccgccaagctgagcggggggaggggggagcggctgtgaaagttgaggaaattgacagactgagaggtgaatggggggacaggcaccaggtcctggctggagggaattggaaaacaaaagtgcatccttctcaccctgcctgggtggagggaatcctgagggaggtagtttgttttgcccatggaaaccccaaagaattatttttcccccttcaatgttgattgcaatattatttcacatttccgtgtatctgttgttttaatctctttcgtctgcgctaatctcgaagtggattattaatggggaagacagagaaggagagattcatttgcaggacttattttgctgtgtgaggagccgaaagccagcagcccccagtcctctctcgacttttctctgcgtcctgtgctcctgcatcctcctgatcccacgatccccattgtccccgaactggccctcggactgagggagtgttcggaacaaatttatattccggcttcctcccgaccccatcacctctgccacgttgggaactctctcggccaaatgcctggggaggggcgtgccagtcaggaaccaggggcaaggcaacctagccgcagcgctcacccctcgcggagacccttcccgtgccaccatccaaatatctaccttcatgggaacctggaagactgctttttcaccccaaatcaaatactggctagtcggaactcccagtgataaatgttggggactgcctcctgccacttgagcaattcctgagtccagggtaatcagatgattaggtgtttttaatggaaatacttagggtttttttttttttttttaagtcagatcctaagtaacctgacatttaatatacataataaagacaagtgttttgttagctattcgattggatcctttgacctgaggttatgcttgtgtgtgtgtctggtctgtaagcctccaatggctacctgggtgcctgaagaggagtcttgcctgtaataactctatgcaaagggctccttcttccttaggagtggtggaagggtggctgtaaagcaaacgtctagagaaagctggattccaatcttttaaattaaaaaagaggaaaaaaaggaagaggaagaaaaatggacatgaacagtggtgctaaatctcagaagaggaacttgataagtcacacatatatttagtatagatatataataatgaaagaaatgtaaaatataaaattaagaatgtgcctgacccatgttgaaaatctttgattttatttattaataatttttaagctgaagatcctgaatgcctttcttctatagacactttatctgtgatggggtggggggagagagttaatttatccagcagctattttcattataataaatacttaaattaggaaagaggtgagtaaccaactgtgaaaatctactttcttgcatcctagaacaagatctagctgtggtcgtgctaggtagctattaaccacccccaccccactcccccgccccgcaaaaaaaaatacaaaatacaaagacgtatacctctgggagtgtggatgtggcaacccatgtggtggcttgtccctcctttgagtgcaattgccagtgactcttgcattcaaagtcttgctttaggcacacattcaaagcacagttcaaagcagtttatcttaatatagtgttggatcagggtattatttgtttcagatttcatcagactggaaacaggagggtgtaacatatttaacaatattaaagcttaaaaattggtgtgaggcagctctctggtcctcttaacacctccacctttacctttccgaagaatcctaaatgctgtgaggctagaagtttatttgtgctctgaggttctcaagcactttcactctttaggaaaaaaaaaaaagtctcacagttatttaagaaattgagcctgaaccatccgaggataggagatttcttccttttccgataaatgcctgtgctcagcatcctcaaatttggctgtctttatttacttctatattagttccaattatgtgcagtgtaaaccagctttttgattaacaaaacagcagagcattggttaagaacacttaaacttcaaaaaaattcccctgggaagtgggctctgaattggttaataatgtgcagtacttgctaaattgctgacttccagatgtggaaaatatctttcttgtttaggacctatgtaacctgattggattacgacagaagcgtcacgttggaagcttttgagtgattatttaattaaccatacagtagaaagctgtattctccaggaaatcccttccatatttgcataaccaatcccttcagagcaaaggtggagtcttttctttttaatcttactttaattgtaatatcaaaaaatatatactccaaaacttagaccccatgccgtttaatatcatgctctcccttccctgctaagtttctctatggcctttctcgtttctcctcctgcctccctacacactctcccttactttgtagtgaggtctccccgaggtgtagagagattcagagatcggccaaccgagtgttctattttaatttacttagagaccctttattaaaatgccaaactactttttaatattgggatccagtccagaaattcatcatgcacacaccctaatacacatgccctaagatgcagctcccatgcagcatttttttttctggctctggtacctaaaaagaaaaaaataacaataagagaaagggcagagaactttgttccagaagctgtactgagatacctacacaatttgatgtgcatctcaaatctggtcattagagatatctgtataagaagagactatctggattgaggacccgggatctttccctttaactttcgccccttggagttctccagttctgtgaatggtgtgcaccgttttccgccctgtactctgtaggatttagtgatgaggataatgatgccaaaggcttgacgggcggggaggggggggtggagggggggagaagggagggaggggggagggaggcgaaggcgaaagggagggagaggaggaagggagggaggtggaatttcatttcttccactaaagcgtttgcggagacttcaaggtataatctatcccagatcctttcccagagagaaacttggcgatcacgttttcacatgatgctcacgctcagggcgcttcaattatccctccccacaaagataggtggcgcgtgtttcagggtctctcgtctctctcctacagaaaagaaaaagaaaaaaatgtcattagaagaggcgtaacacgtcagtccgtccccaggtttgtgtttcctggagtggccgaaagagatcagttctaacctgctctgcaggaataacggtcctgcctcccgacactcttggcgaggtttttgtacagtttgctccgggagctgtttcttcgcttccacctttttctcccccacacttcgcggcttcttcatgctttttcttctcaccatttctggccaaaactacaaacaagacttcgcag

SEQ ID NO: 5 - DNA, 3850 bp, homo sapiens
atcctagaagctttgaaaatatatccactgttcagagggacaacaaaggcagttagactgtcctgaacggtcctgcctcaggctgaaatttttgtagcacttgatcagttgcaaagtgatcttccctttaatatctcattttatcattgggtatctgaagaggaagtggaattggggtaagaatttaggttcttgccatagcatttgggtggccagggtaagcctcagggtggaggacccttaaagaaaactctaaggattttaaggagagtcaaactctacattcatccaggcaaacatctactcttccattgattaatggatccactcatccgtgcaacacattcactctttcatccatccattcatccatctatcctccatcaatccatccatgtatctttcattcatccatttatccatttatcctccatccatccgtccatccaccctccatccactcctccatccatcatccatccattcttccatccatcccctatccatccatccacccatctattcatccatccacccatcttccatccatgcatccatcttccattcacccatccatcaatccatcccctatccatccatccacccatctattcatccatccatccatcttccatccatgcatccatcttccattcacccatccatcaatccatctacttatccatcctccatttatccacacatccattgcacataaagctgcgaatgaggtggtcatagcccctgccttagtggatctcaggctagttagtggagataaaattctctgattggcgcagtctgggtcattcatccacttcgttgcttggggaaggtggggcagctgggttgatagctctaccaagattatgtttagagtagtttccccaaggaacacactggggtgctgttatgagaagaagggaggaattattctgggtaggcacaatcaacaaatgtccactagatttagtggcacttaataattatgggatggagggacgagaaaggatgtggggaggcattcctagcttttctccttttctaaatcacaggggaagcctggttttcttgtttattggccaaaatttcatcatctggaccttgggtccctgtcataaggacccagaagaatccatctctttctagatacctgtctataagtatcctagctggggtcttcactcttccttcctccacctaaatgcatcagctgcccccaagatgcttgttggatacacatggcacagaagtgcttatagttaggaaacaaaggctggaatttctttatgctctgacaaccaggaggtgctgcccaagactctgaaatgattcctgagccagcccccaggcccctgcagtagagtttgagtctaaaccaaagtggttctcatctctggccacacattggaagcatctggtgagcttttacaaaatactgatgcctaagtcccaaccccaaacattctgatcaaatcgatgtctggtggtgctcaggtcgttgaatacaaattttaacagctctccaggtgattctaatgtgtagtctgggttaagaaccactggtctgagtgcaattttgaaaaccccgttcataactgtgctactttgctttcagggcataagcatgatgaagggagaggctgtggcttgttgatggttgcatccccaacatcttgtggcagtgccaggcaggtgctcaataaatatttgtggaatgatggatggcttggcacaaatccccttaatggagctgagatggctcccctgaagaatgcgtgtcctcaattcctcctggcacatggggaaggattcgaatcacttgaggtcaagctgtggttctcacgaggatcttaagacctctcccgtctaatcttctcatcctcaacactggctgcaccttagattcatctgaagtttaaaaaaattttcaggccgggcgtggtggctcacacctgtaatcccagcacttggggaggccgaggcgggtggatcacgaggtcaggagatcaagaccttcctggctaacacagtgaaaccccatctctactaaaaatacaaaaaagtagccaggcatggtggctggcgcctgtagtcccagctactcgggaggctgaggcaggagaatggcgtgaacccagaaggcagagcttgcaatgagccaagatcgcgccactgcactccagcctgggcgacagagcgagactccgtctccaaaaaaaaaaaaaaaaaaaaaaaaaaaaattaggaattctgcttcagtgggtttggattgcagcccagcacgggagtttaaaaaggtgtcccaggtgattctaatatgtgggaagggttgaagaccactgttttgatcttttctacctctgtgagtctgtttccaacttcagctaagttggtggagccccctggcagagttttattggagggtgttctttagttactgggtccttatgcagtgtttgccaaatgtacctgttcattaatgctcacgttgggtgcttaattaaaaatacacattcacgagtctgtattcagacttatggaatcagaagctccagggaggggcctgggaatcatgctttaaacaagcagccaggcgtgtttgtcacttgccaaccgccccatcagaactgtactcctcgttctccctccagcacacccagccccttcgagctctggctctgtgttttggttctcactgccagaacacacttccccaagatcgcctcatcaatcaggtttcttcaaatgtcacctcctcagagaggtcctccttgaccacccaagggactaaagccaccccaacctttatcccataccctggtgagttctctcctggccccataagcagatgctttttgtcctgtttctctccttcaggcctctcccactaccagagcctgagcttccgagggcaacagtgtctggctccggctgggtgcccagcacctggcacgtggtaggtggccaggaggcatagctgtaacgggggggactagtgctgtcagtccgattccttcctaggcgctggggagttgagttcagggccatgcaggtgtctgaagtactgtgaaaatcatgccgcccccgtgggcacacggagacgcctggagacacgggtctgcaatagaagggggtggatacccccttccaccccatggagaagaaaagcaggaaaaagtgtggggccagtgggtcccttagggcaaaagtcctagtggcggcagctttcttgtctagaccctggttcgcgcagcgcgtgtcgcactcacccgtggagggtgggagacagcggccgccccagcgcccagcggtggcccgggacagtgtggtcaggtccgcggacagcctccttcctcccggcaccggccagcccaggccgcgcgcccctcccctccggtcccctggtggcggccccgcccccgccggcctggccgccctgtccccgcctccacgccccccccccccccgcctccccgggctgcaacaggtgcctccggagcaggaagctcgcgccgccgtcgccgccgccgctcagcttccccgggcgcgtccaggacccgctgcgccaggcgcgccgtccccggacccggcgtgcgtccctacgaggaaagggaccccgccgctcgagccgcctccgccagccccactgcgaggggtcccagagccagccgcgcccgccctcgcccccggccccgcagccttcccgccctgcgcgccatgaacgcccccgagcggcagccccaacccgacggcggggacgccccaggccacgagcctgggggcagcccccaagacgagcttgacttctccatcctcttcgactatgagtatttgaatccgaacgaag

SEQ ID NO: 6 - DNA, 4044 bp, homo sapiens
  misc_feature (3503)..(3503): n is a, c, g, or t
cagtttgtgcggccccaccataagcagcagtttgaggaggtctgtatccagctgacaggacgaggctgtggctatcggcctgagcacagcattccccgaaggcagcgggcacagccggtcctggaccccactggtaaatggggccccaggtgggaccctcagactcctgcgtggaaggcagtgtgggccagagtcctgggctgcttggggtgggcatcctcgggccctgcttggccccgcctctctgttcccctatgggagtgatgggggcctccacctccaccaccagcaccagcagcaccacctccacctccacctccacctccacctccaccaccacctccacctccaccaccacctcctccaccaccaccacctccaccaccaccaccaccaccaccacctccacctccaccacctccacctccaccaccaccacctccacctccaccaccaccacctccacctccaccaccacctccaccaccaccacctccacctccaccaccacctccaccaccaccaccaccaccaccaccaccaccaccaccacctccaccaccaccacctgcaccaccacctccacctccaccaccaccaccacctccacctccaccagcagcagcatcacttgttggggagaccctgtgcaactccatgcacagccctgtccctgccatagccccgacccctaagcacagccctgtccaactgccacacgtcccctgcctcccatgcatggtcctggggggtcaactgcacacgccagggtcctagggtcctagacccctgtcctccctgtttctgcctctgtttggggtggagtccaagtctccagaggcggaagcatctgtgttcgtgtgttaatgaacagcccctacagagttcccctagttcacccaggggggaacctagcctgttgggacgaccccagatcccttctgggcttggtactcactgggatatcctcatgcctgcacccagcctacggctctgagctcctgagtggggctttggcctgcccgccactgttccagcccccatccagcaggctggtgtctcctctgatgcccccagcacccaggcgtgtacctgcctgggttttcccgccctggtctgaggtgggtgaggcctggcctccctagccagccctgcccccccaccccagggaactttccagatgctctcgaccagctttgtggctctacatctcttcatcaggaagaacggcgccggatcccaagctgaccgtgtccacggctgcagcccagcagctggacccccaagagcacctgaaccagggcaggccccacctgtcgcccaggccacccccaacaggtagctgactcctgaaccgtgtgcagcctacgacttggtgggtccctcagtggcttcacgaggctaactcttgagtgtggccggggctgcccctgtggggagccatctcatggtggggactgctcccggttctgcaccccgcagttgtcctgagcagctctccaggagttcctggaggaagggcgggcagggcggtgggactctcagtcctccaccccagcgccactctgagccatgctactcccacaccaggagaccctggcagccaaccacagtgggggtctggagtgcccagagcagggaagcagggccagcacgccgtgagcgcctacctggctgatgcccgcagggccctggggtccgcgggctgtagccaactcttggcagcgctgacagcctataagcaagacgacgacctcgacaaggtgctggctgtgttggccgccctgaccactgcaaagccagaggacttccccctgctgcacagcaagtggccctggcgtggggaacagccggtggggtggggggcaggggacaaaatgggggctgtgccgggtctgattgaagctccccgcagggttcagcatgtttgtgcgtccacaccacaagcagcgcttctcacagacgtgcacagacctgaccggccggccctacccgggcatggagccaccgggaccccaggaggagaggcttgccgtgcctcctgtgcttacccacagggctccccaaccaggtagggcacctgcctggctgctcctggcagcgccccaaccgcacgcagccctgggagtgagcagcaaagccccaggcccccctcagactcaagtctctgtctccaggcccctcacggtccgagaagaccgggaagacccagagcaagatctcgtccttccttagacagaggccagcagggactgtgggggcgggcggtgaggatgcaggtcccagccagtcctcaggacctccccacgggcctgcagcatctgagtggggtgagcctcatgggagagacatcgctgggcagcaggccacgggagctccgggcgggcccctctcagcaggctgtgtgtgccagggctgtggggcagaggacgtggtgcccttccagtgccctgcctgtgacttccagcgctgccaagcctgctggcaacggcaccttcaggttggtgcctggccactacagttcctgctgggtgtagccccaggtgatgggctgagggggaaagggcaggcccttgtcctggtggcaacgcctggcagacgtgtgcagtgggccggttgtctcacaggcctctaggatgtgcccagcctgccacaccgcctccaggaagcagagcgtcatgcaggtcttctggccagagccccagtgagtgcccacggaggcccccagcacacccaacgtggcttgatcacctgcctgtccagctctggtgggccaagaacccacccaacagaataggccagcccatgccagccggcttggcccgctgcaggcctcaggcaggcggggcccatggttggtccctgcggtgggaccggatctgggcctgcctctgagaagccctgagctaccttggggtctggggtgggtttctgggaaagtgcttccccagaacttccctggctcctggcctgtgagtggtgccacaggggcaccccagctgagcccctcaccgggaaggaggagacccccgtgggcacgtgtccacttttaatcaggggacagggctctctaataaagctgctggcagtgcccaggacggtgtcttcgtggcctgggcttggtggtgggagttgagggacagggagttggcagaggcccctcccagcctgccatgtgacactgtacttcctccacggtgggctcagccctgccctcatcctcacagccgcagccaagctgcagttggtaggggatccaccgacacaccaggctgcctgggctggtctctgggttgggagctgccccaggtgctgaggagggcagctccctggctggtgaggcccctcccagaaccacccttggactgagctctggggagggatggtaccaggtgggtgaggggggctgcctggggagggaggggttcctatggggcgtggcgaggctggcccagccctctccccgcccatatatgtagggcagcagcaggatgggcttcmrnatggacttgggcggcccctccgcaggcggaccgggggcaaaggaggtggcatgtcggtcaggcacagcagggtcctgtgtccgcgctgagccgcgctctccctgctccagcaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctagagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccg

SEQ ID NO: 7 - DNA, 3991 bp, homo sapiens
gctgggattacaggcgtgagccaccaagcctggctaattttgtatttttagtagagacggggtttcactatgttggtcagcctggtcaccgaactcctgacctcaggtgatccacccacctctgcctcccaaagtgttgggattacaggtgtgagccactgcacccagccatgtgctcttatccttagatataaaacctaccaagttttctttctgggagtgattctggctcagggtgtccccacagagacctggaagtggccctcaggagggatctctcttccagagcttcagcccagccccaaccagacttccacttcctgaactgaaagccacatgggaacaccagccaaaaactgggaagagttccactggttcatcctcatcagaatcatcattttagtttatgaataaaaatacaaagcttctgctaattttagtcccataatgcctgggagctatagcaaccaatcctataaaaatatacatctgtccggatggcaaaacagatatgtggagagtccaccagcaccctgtattcagcagccaaaagcacttggcttttgaaccagggtgagaggccttggggggtctttatttatttatttagagacggagtcttgctctgtctccaaggctagagtgcaatggtgccatctcggctcactgcaacctccgcctcttgggttcaagcgattctcctgcctcagcctcccatgtagctgggattacaggcacgtgccatgatgcccaactaatttcttttgtatttctagtagagacagggttttgccatgttggccagcctggtgtcgaactcctgaccgcaggtgatctacctgcctcagcctcccaaagggctggaattacaggagtgagccaccatgcctggccttattattattattattattttattattattacagacagcatcttgctctgtcaccgaggctagtctcaggcttctggccttaagcaatcctcctgcttcagcctcagagtagctgggactccaggaacatactactgcacccggcccttgggggtcatttggagaagactccttcttgggtgtttgtggatttttctgcagagaaagttcattgcttcctttaggttctcaaaaggaccaaacaccctaacttttatattccactttagtggtttgagggtctcttgggtgtggggcagtgttccaggctggaaccctagtaaaagacaaccctctcttttttcagatgggcaagtcggaagttcatatatttattgaataaaatattgcaagccagctttgtgtggggcactggtcaacatgctgggaacaaagcagtaaacagacatgtgaagtccctgctctcaccaagcttctgtctagttggggaaacaaatggtttctgagaatgacaggtgatggggaggaaataaaacagtgtaaggcgatgaactgtgcctgtgagagaggcaagtagattgagtggccctaaggaggtgacattagaataggcacaggaaaatcaggtggaagagtgtctctggcagaggtaagagcaaatgcaaagatactgggcatggtggctcagacttgtaatcccaacacttagagaggccaaggcgggcggatcacttgtggtcaggagttcgagaccagtctggccaacatggtgaaaccctgtctctactaaaaatacaaaaattagcctggtgtggtggtgcatgcctgtaatcccagcttcttgagaggctgaggcagcagaatcacttgaacccaggaggtggaggttgcagtgagttgagattgcgccacagccctccagcctgggcaacaaagtgagagtctgtctcaaaaaaaaaaaaaaaatcctggggtgggagaagctttggggtagttgaaggactggaaggaagttggcatggctggagcagcgtgggcacagaggacagtggtaggaggagagaatggcaagagagacaggggccacagcggatggaagtgtatcatgagggtgttctctgccttcttcctttccatggtattcaaacccaacccatgcttccaggtccacctctttcaggaatccaccaatctgaagccctgggataagccttagagtctgtaccactcatgtaagtgggtgtatgtgtaaggcacaacctttcagcaagatccaaagctccttgaatagtgagttatctcgattgattgttcacagtcacagattgaattccttgtactttttttcccttctcagttctgcacttaactcgtctaaaaaaattaaaaaagaatttaagaaaccacaaagctaagctgggtgcggtggctcacgcctgtaatcctagcactttgggaagccaaggcattcggattgcccaagctcaggagttcgagaccagcctgggcaacatgttgaaaccccatttctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccaggtatttattaattctcatacctcccagagtgttaggcacaaaataaacattcaaccaagacctgttgcactgagcagttcatatataacaggagtgacccaagttgaaacgtagaatcagccctctcataccactttttgccaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcaattaccttatctgttcaatactggtggcacaggtccaccagctagatgtctaatcccttatgtgtctattagtggtacaagtggagtttgagtgggatttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctgcccctgggggccattgggtaattccttgcagtctccaggcctcacttgccctctgaacaagaaagaggctgttctgggtcatccctccaggcctgtccagccctggcactctgtgagtcggtttaggcagcagccccggaacagatgaggcaggcagggttgggacgtttggtcaggacagcccaccgcaaaaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgaggtatgtgtgacccccgcccagcctttcccttctatagttgcaccaaccccgacacccccgttcacgccgtcagctcgtgtgcaagggagggaagctctgctgaggatgcgcctctcctcccggctccatcacggctccccttaagagcatggccctcggtcctgtctgcctgttgcttttcagaaggtggactcactgtgtaactttgtcttcccttacaggtttacagaaaataatctcactatgtcttcgggggagcattttctcactctctgtttttctctgtgtctgtctctggttt

SEQ ID NO: 8 - DNA, 479 bp, homo sapiens
ggactcgggagtggtgaaacgcctgtacacgttggatgggaaacaggtgatgtgccttcaggacttttttggtgatgatgacatttttattgcatgtggaccggagaagttccgttaccaggatgatttcttgctagatgaaagtgaatgtcgagtggtaaagtccacttcttacaccaaaatagcttcatcatcccgcaggagcaccaccaagagcccaaggacgtccaggcgtagcagtcccctgcctccaccagctcagttaatggaacccctggtagtcagctctctactccgcgctcaggcaagtcgccaagcccatcacccaccagcccaggaagcctgcggaagcagaggagctctcagcatggcggctcctctacgtcacttgcgtccaccaaagtctgcagctcgatggatgagaacgatggccctggagaagaagtgtcggaggaaggcttccagattccagctacaat

SEQ ID NO: 9 - DNA, 254 bp, homo sapiens
aacactaagactgtgtccatgttagaactcatagaagttaatggaacccctggtagtcagctctctactccgcgctcaggcaagtcgccaagcccatcacccaccagcccaggaagcctgcggaagcagaggagctctcagcatggcggctcctctacgtcacttgcgtccaccaaagtctgcagctcgatggatgagaacgatggccctggagaagaagtgtcggaggaaggcttccagattccagctacaat

SEQ ID NO: 10 - DNA, 1504 bp, homo sapiens
  misc_feature (1403)..(1403): n is a, c, g, or t
aagcaagtttgccactgtccatgcttacaggattcatgtacacctgggaatatacatatgacatgagttgagtgaaaatttagtaacacaaattagaagaaagagaaagatgaaaattagtttaaaggaggtaatttacttaaggaggaaagttttgcattgaggcataaggaagtgacttccattaattggtgtgaaatggggagaattttatgtgcacaggcatagagacggacatgtttaggtgaaggtgaagaaaattagttcaagtgaccttgattagaattgaaatgtccaccgtagattatagagatagatgaactaactatgaactgatgattagggtgtaaagagatatctccattgccagatatagccttatactgtggacaagcatatttttcctgcagagatttatcaaagacctataaatggtatttgcccctctttcaaatatgacacaaaaattactgcttagatagatccagaaatacattggaaagttttgtggatgaggatctggaaagtatacacataaaagtctaaattagaaatatttatactaatctttagagtaatgcagaaaaaatgaccactttctctggtactcttttgctattctatgtgataaactctttctcttccagtttcctaaaaagttatgtcatgaaaaggctgaatttttctctagaaaaatctgctctgtctttcccaggcagcataaaaattggaaatctcacaagaaagcacctctggcttgctaaagaaatttgtgagaaatggaaacagctatagataagtatctaacttttaggaagccattcaaacattgctgaaatactttgtcttttgatatttgcctgaaacttaacttctaggacccagggtgggtggatgagtcgaaggggcatacggtggcatttctttccgtgagtctcttacagtctcctatttaaattgagttaggattacttctggcaaaatcccaacataaaaatcttctaggaagatcagttctgtaaattagacatactagataaatgggcatcaagcagtttttcaaaattatgcagttgttaacttcataaggggaaataaaaatgtatgcatttacattgtatgtattaaaacaaggcagagcatttctatacgttcctaagttatacaaacatatatgtaagagtgaaatatgtaaaaaaacttttacataagcagatgcatacaaactccagatgtgctctttttcttactgtgggttgtgtcttctataagggaaaaagaaatatttatcatttcttttactgctgaggggatgggtgcggctctcatatttcagaaaggggctggaaagtgagggaagccaaactttttcctatttaaggccaaagcaaaggaatctcmrnaagtggctgagttttatgacgggcccggtgctgaagggcagggaacaacttgatggtgctactttgaactgcttttcttttctcctttttgcacaaagagt

SEQ ID NO: 11 - DNA, 21 bp, artificial sequence
  synthetic oligonucleotide
ggagtggtgaaacgcctgtac

SEQ ID NO: 12 - DNA, 20 bp, artificial sequence
  synthetic oligonucleotide
ggttccattaactgagctgg

SEQ ID NO: 13 - DNA, 29 bp, artificial sequence
  synthetic oligonucleotide
acactaacactgtgtccatgttagaactc

SEQ ID NO: 14 - DNA, 21 bp, artificial sequence
  synthetic oligonucleotide
aagccttcctccgacacttct

SEQ ID NO: 15 - protein, 729 aa, Homo sapiens
MSFGRDMELEHFDERDKAQRYSRGSRVNGLPSPTHSAHCSFYRTRTLQTLSSEKKAKKVRFYRNGDRYFKGIVYAISPDRFRSFEALLADLTRTLSDNVNLPQGVRTIYTIDGLKKISSLDQLVEGESYVCGSIEPFKKLEYTKNVNPNWSVNVKTTSASRAVSSLATAKGSPSEVRENKDFIRPKLVTIIRSGVKPRKAVRILLNKKTAHSFEQVLTDITDAIKLDSGVVKRLYTLDGKQVMCLQDFFGDDDIFIACGPEKFRYQDDFLLDESECRVVKSTSYTKIASSSRRSTTKSPGPSRRSKSPASTSSVNGTPGSQLSTPRSGKSPSPSPTSPGSLRKQRSSQHGGSSTSLASTKVCSSMDENDGPGEEVSEEGFQIPATITERYKVGRTIGDGNFAVVKECVERSTAREYALKIIKKSKCRGKEHMIQNEVSILRRVKHPNIVLLIEEMDVPTELYLVMELVKGGDLFDAITSTNKYTERDASGMLYNLASAIKYLHSLNIVHRDIKPENLLVYEHQDGSKSLKLGDFGLATIVDGPLYTVCGTPTYVAPEIIAETGYGLKVDIWAAGVITYILLCGFPPFRGSGDDQEVLFDQILMGQVDFPSPYWDNVSDSAKELITMMLLVDVDQRFSAVQVLEHPWVNDDGLPENEHQLSVAGKIKKHFNTGPKPNSTAAGVSVIALDHGFTIKRSGSLDYYQQPGMYWIRPPLLIRRGRFSDEDATRM

SEQ ID NO: 16 - protein, 422 aa, Homo sapiens
MLELIEVNGTPGSQLSTPRSGKSPSPSPTSPGSLRKQRSSQHGGSSTSLASTKVCSSMDENDGPGEEVSEEGFQIPATITERYKVGRTIGDGNFAVVKECVERSTAREYALKIIKKSKCRGKEHMIQNEVSILRRVKHPNIVLLIEEMDVPTELYLVMELVKGGDLFDAITSTNKYTERDASGMLYNLASAIKYLHSLNIVHRDIKPENLLVYEHQDGSKSLKLGDFGLATIVDGPLYTVCGTPTYVAPEIIAETGYGLKVDIWAAGVITYILLCGFPPFRGSGDDQEVLFDQILMGQVDFPSPYWDNVSDSAKELITMMLLVDVDQRFSAVQVLEHPWVNDDGLPENEHQLSVAGKIKKHFNTGPKPNSTAAGVSVIALDHGFTIKRSGSLDYYQQPGMYWIRPPLLIRRGRFSDEDATRM